(19) United States
(12) Patent Application Publication
(10) Pub. No.: US 2022/0325297 A1
(43) Pub. Date: Oct. 13, 2022

(54) RECOMBINANT ONCOLYTIC NEWCASTLE DISEASE VIRUSES WITH INCREASED ACTIVITY

(71) Applicant: Arno THALLER, Markt Berolzheim (DE)

(72) Inventor: Arno THALLER, Markt Berolzheim (DE)

(21) Appl. No.: 17/642,717

(22) PCT Filed: Sep. 2, 2020
(86) PCT No.: PCT/EP2020/074404

§ 371 Date: Mar. 14, 2022

(30) Foreign Application Priority Data
Sep. 19, 2019 (EP) 19198308.9

(51) Int. Cl. C12N 15/86; C12N 7/00; C07K 14/005; A61K 35/768; C07K 16/28; C07K 16/22; A61P 35/00; A61K 39/00

(52) U.S. Cl. CPC C12N 15/86; C12N 7/00; C07K 14/005; A61K 35/768; C07K 16/2827; C07K 16/22; A61P 35/00; A61K 39/0011; C12N 2760/18121; C12N 2760/18122; C12N 2760/18132; C12N 2760/18142; C12N 2760/18152; A61K 2039/5256; A61K 2039/585; C12N 2760/18171

(57) ABSTRACT

The invention relates to transgene expressing Newcastle Disease Viruses (NDV), which have been demonstrated to possess significant oncolytic activity against mammalian cancers and/or an improved safety profile. The invention provides novel oncolytic viruses through the use of genetic engineering, including the transfer of foreign genes or parts thereof, such as genes encoding Atezolizumab or Bevacizumab. The present invention also provides nucleic acids encoding a reverse genetically engineered (rg-)NDV comprising one or more of these foreign genes and having a mutation in the HN gene, said mutation allowing replication of said rgNDV in a cancer cell to a higher level than replication of an otherwise identical rgNDV not having said mutation in the HN gene.

This invention relates to recombinant Newcastle Disease Viruses (NDV), which have been demonstrated to possess significant oncolytic activity against mammalian cancers, especially selected from the specific cancers as described herein and/or mentioned in the claims. The invention provides the development of novel and improved oncolytic agents through the use of genetic engineering, including the transfer of genes across species boundaries in order to produce improved genetically modified viruses carrying these transgenes.

### BACKGROUND

NDV is known as an oncolytic virus that is a virus for use in oncological treatment, preferably in the treatment of human subjects in need thereof. A number of RNA viruses, including NDV, reovirus, measles virus, and vesicular stomatitis virus (VSV), are members of this novel class of viruses being exploited as potential oncolytic agents. These oncolytic viruses are characterized in inherently replicating selectively within and killing tumor cells while leaving non-tumor cells unharmed. Accordingly these oncolytic viruses offer an attractive new tool for cancer therapy.

NDV derives its name from the site of the original outbreak in chickens at a farm near Newcastle-upon-Tyne in England in 1926. The virus is an economically important pathogen in multiple avian species and it is endemic in many countries. NDV is a member of the Avulavirus genus in the Paramyxoviridae family and is a non-segmented, negative-strand RNA virus, whose natural host range is limited to avian species; however, it is known to enter cells by binding to sialic acid residues present on a wide range of human and rodent cancer cells. The oncolytic property of NDV, i.e. to selectively replicate in and destroy tumor cells, while sparing normal cells, is believed to be based in part on defective antiviral responses in tumor cells. Normal cells, which are competent in launching an efficient antiviral response rapidly after infection, are able to inhibit viral replication before viral-mediated cell damage can be initiated. The sensitivity of NDV to interferon (IFN), coupled with defective IFN signalling pathways in tumor cells, provides one plausible mechanism whereby NDV can replicate exclusively within neoplastic tissue. The selective replication of NDV in human tumor cells may also be caused by several other mechanisms, including defects in activation of anti-viral signalling pathways, and activation of Ras signalling and/or expression of Racl (Schirrmacher, 2015, Expert Opin. Biol. Ther. 15:17 57-71).

Due to its tumor-specifity NDV is already under investigation as a safe clinical oncolytic virotherapy agent (Cassel et al., 1965, Cancer 1:863-888; Steiner et al., 2004, J. Clin. Oncol. 22:4272-4281; Schulze et al., 2009, Cancer Immunol. Immunother. 58:61-69; Csatary et al., 1999, Anticancer Res. 19:635-638; Pecora et al., 2002, J. Clin. Oncol. 20:2251-2266; Lorence et al., 2003, Curr. Opin. Mol. Ther. 5:618-624; Freeman et al., 2006, Mol. Ther. 13:221-228). The safety margin of NDV is a function of its inability to replicate and kill normal human cells.

Typically, an oncolytic NDV strain is defined as an NDV for use in oncological virotherapy, preferably in the treatment of human subjects in need thereof. Multiple preclinical model studies have shown significant anti-tumor activity of natural and recombinant oncolytic NDV strains after varying treatment modalities. Promising results were noted in preclinical models of glioblastoma multiforme, anaplastic astrocytoma, leukemia, lymphoma, melanoma, neuroblastoma, osteosarcoma, rhabdomyosarcoma, Ewing's sarcoma, fibrosarcoma, pheochromocytoma, colon carcinoma, lung carcinoma, prostate carcinoma, breast carcinoma, ovary carcinoma, gastric carcinoma, mesothelioma, renal cell carcinoma, and head and neck carcinoma. One typically useful oncolytic NDV that has been extensively explored for use in the treatment of humans, and for which an advantageous safety and efficacy profile has been established in human trials, is a strain named MTH-68/H (Csatary L K, et al., J Neurooncol. 2004 March-April; 67(1-2):83-93). MTH-68/H therapy has been employed in a range of different tumors with success. MTH-68/H has been developed into a highly purified, lyophilized product, containing live, replication competent viral particles, grown to standardized titers. A Phase II clinical trial in humans was completed where the inhalatory mode of administration was used on patients suffering from a variety of advanced malignancies no longer responding to conventional cancer therapies. The study demonstrated relative efficacy, an overall improved quality of life, and a relatively benign side effect profile (Csatary L K, et al., Cancer detection and Prevention, 1993, 17(6):619-627). Success of treatment with MTH-68/H has also been shown in glioblastomas and anaplastic astrocytomas. Glioblastoma multiforme (GBM) is the most common primary brain tumor and by far the most aggressive form of glial tumors. This neoplasm has a poor prognosis, averaging six months to a year without therapy or about one-and-one-half years with conventional therapy. Four cases of advanced GBM and anaplastic astrocytoma were treated with the NDV viral product MTH-68/H after the conventional modalities of cancer therapy had failed. Results included survival rates of twelve and half to six years post-viral treatment for the surviving patients, and six years for one patient who has since died, after having discontinued treatment. Against all odds, these surviving patients returned to good quality of life and to a lifestyle that resemble their pre-morbid daily routines including giving birth to a healthy child and full professional activity. They have been able to enjoy good clinical health. These patients regularly received the MTH-68/H viral therapy for a number of years without interruption as a form of monotherapy once the classical modalities failed. The MRI and CT results have revealed an objective decrease in size of the tumors, in some cases the near total disappearance of the tumor mass.

As reviewed by Zamarin and Palese (Future Microbiol. 2012 March; 7(3): 347-367), when compared to other oncolytic agents in development such as poxvirus, HSV-1, adenovirus, measles, and reovirus vectors, NDV as an oncolytic virus exhibits several advantages over other viruses for use in oncological treatment.

The first advantage is that NDV is an avian pathogen. This avoids the problems of preexisting immunity that can neutralize virus infectivity and pathogenicity of the virus in humans. These potential problems exist when using vaccinia, HSV-1, adenovirus, and measles vectors. Based on serological studies it seems that about 96% of the human population is seronegative for NDV, which avoids the problem of pre-existing immunity. The actual level of seropositivity in today's population may be even lower due to low human exposure to NDV, as the outbreaks of which are primarily limited to farm settings. To attest for viral safety, administration of virulent strains to humans has been shown to result in only mild to moderate adverse effects, with mild conjunctivitis, laryngitis, and flu-like symptoms as the only reported side effects. Natural human infections with highly virulent (avian) strains have been reported in the literature in farmers and laboratory workers and have been limited to mild symptoms such as conjunctivitis and laryngitis.

The second advantage offered by using NDV as an oncolytic agent is that NDV, similarly to other oncolytic viruses, possesses strong immunostimulatory properties that are the basis for oncolytic therapy being considered an immunotherapy. These properties include the induction of type I IFN and chemokines, upregulation of MHC and cell adhesion molecules, and facilitation of adhesion of lymphocytes and antigen-presenting cells (APCs) through expression of viral glycoproteins on the surface of infected cells. These properties have been shown to generate effective anti-tumor immune responses, which may persist long after clearance of the primary viral infection.

A third advantage is that the NDV genome has the plasticity to enable the incorporation and stable expression of foreign genes of relatively large size. Since these viruses replicate in the cytoplasm of the cell and not in the nucleus, an unintended integration of the foreign gene into the host genome is avoided. Such an unintended integration of foreign genes carried by an oncolytic agent into the host genome could be a safety problem with some DNA oncolytic vectors. Moreover, the absence of homologous RNA recombination ensures that foreign genes incorporated in and expressed from the NDV genome are stable for many serial passages in cell culture and in tumor cells.

Lastly, the ubiquitous nature of the NDV receptor allows for utilization of the virus against a wide variety of tumor types. The specificity of NDV for cancer cells due to their defects in antiviral pathways ensures viral safety.

Nevertheless, the full view of all clinical data generated to date in different trials of different NDV strains shows that there is much need for improvement in terms of percentage responders and magnitude of therapeutic effect. Also it is desired to further improve the viral safety. This need for improvement has spurred efforts to design improved recombinant NDV strains, which is the subject of the present patent application.

Similar to other paramyxoviruses in its family, the 15186, 15192 or 15198 nucleotide(nt)-long negative single-strand RNA genome of NDV encodes six genes including the nucleocapsid protein (NP), phosphoprotein (P), matrix protein (M), fusion protein (F), hemagglutinin-neuraminidase (HN), and RNA-dependent RNA polymerase (L) (Lamb R, Paramyxoviridae: the viruses and their replication. In: Knipe D, editor. Fields Virology, Lippincott Williams & Wilkins; 2007, pp. 1449-1496). The genes are separated by junction sequences that consist of three elements, known as gene start (GS), intergenic (IG), and gene-end (GE) motifs, which regulate mRNA transcription. In the P gene, a unique RNA editing mechanism adds non-templated G residues, resulting in the expression of V and W proteins that are colinear to the P protein in the amino-terminal end. The genomic RNA is bound in a ribonucleotide protein complex (RNP) consisting of NP, P, and L proteins and is surrounded by a lipid envelope containing three virus glycoprotein spikes, HN, M and F. An important factor that influences the extent of virulence of NDV in birds is the cleavage site of the F protein.

Pathogenic classification and virulence of NDV strains in birds generally correlates with their oncolytic properties in human cancer cells. Velogenic (high virulence) strains produce severe respiratory and nervous system signs, spread rapidly through chicken flocks, and can cause up to 90% mortality. Mesogenic (intermediate virulence) strains cause coughing, affect egg production and quality, and can result in up to 10% mortality. Lentogenic (low virulence) strains produce only mild symptoms with little if any mortality. Correspondingly, velogenic strains can efficiently carry out multicycle replication in multiple human cancer cells with effective and efficient cell lysis, lentogenic strains are more attenuated due to lack of activation of the FO protein, and mesogenic strains convey intermediate effects. On the basis of these observations in human cancers, NDV strains have been classified as either lytic or non-lytic, with velogenic and mesogenic viruses being lytic (high and low respectively), and lentogenic viruses in general being non-lytic. Early studies demonstrated that the lytic abilities of lentogenic NDV strains could be enhanced by the introduction of a polybasic cleavage site into their F proteins (Peeters et al, J. Virol. 1999; 73:5001-5009). Other NDV proteins including NP, P, V, HN, and L have also been shown to be implicated in virulence in birds. A deletion (18nt) introduced in the NP gene (Mebatsion T et al., J Virol. 2002 October; 76 (20):10138-46) resulted in the deletion of NP residues 443-460. The resulting mutant NDV propagated in embryonated specific-pathogen-free (SPF) chicken eggs to a level fully comparable to that of the parent virus.

In addition, a B-cell epitope of the S2 glycoprotein of murine hepatitis virus (MHV) was inserted in-frame. Recombinant NDV viruses properly expressing the introduced MHV epitope were successfully generated, demonstrating that the NP can be used as the insertion point in the NDV genome to insert foreign or transgenic sequences to be co-expressed with NDV during virus infection.

Several studies showed that type I IFN antagonist activity of NDV V protein is associated with pathogenicity in birds. In recombinant viruses possessing attenuating mutations or deletions in the V protein or in viruses expressing no or lower levels of V protein decreased pathogenicity in birds was observed (Huang et al., 2003, J Virol. 77:8676-8685; Mebatsion et al., 2001, J Virol. 75:420-428; Qiu et al., 2016, PLoS ONE 11(2): e0148560. doi: 10.1371/journal.pone.0148560; Elankumaran et al., 2010, J Virol. 84:3835-3844; Jang et al., 2010, Appl Microbiol Biotechnol. 85:1509-1520; Alamares et al., 2010, Virus Res. 147:153-157; Park et al., 2003, J Viro1.77:9522-9532.). It was found to be sufficient to introduce a small (6 nt) deletion in the P gene to abolish expression of the V protein (Mebatsion et al., 2001).

Recently, two studies showed the role of the polymerase complex in viral pathogenesis, with L protein contributing to pathogenicity of the mesogenic Beaudette C strain, and NP, P, and L proteins contributing to pathogenicity of the velogenic Herts strain (Rout & Samal, 2008, J Virol. 82:7828-7836; Dortmans et al., 2010, J Virol. 84:10113-10120). In addition, several studies noted the contribution of the HN protein to virulence in birds (Römer-Oberdörfer et al., 2006, Avian Dis. 50:259-263; Paldurai et al., 2014, J Virol. 88: 8579-8596.) The HN (hemagglutinin-neuraminidase) protein of NDV is a multifunctional protein with receptor-recognition, hemagglutinin (HA) and receptor-destroying neuraminidase (NA) activities associated with the virus. HN is thought to possess both the receptor recognition of sialic acid at the termini of host glycoconjugates and the neuraminidase activity to hydrolyze sialic acid from progeny virion particles in order to prevent viral self-aggregation. It also recognizes sialic acid-containing receptors on cell surfaces and promotes the fusion activity of the F protein, thereby allowing the virus to penetrate the cell surface. Thus, the HN protein plays a critical role in viral infection of birds.

A recombinant NDV vector virus can be provided by a reverse genetics (rg) system, which has been known since 1999 (Peeters et al., 1999, J. Virol. 73:5001-5009). This system allows desired modification and engineering of NDV genomes. Indeed, various reports have shown the successful use of recombinant NDV vectors engineered to express various transgenes, i.e. foreign genes incorporated in the genome of the virus, with the goal of improving viral oncolytic efficacy (Vigil et al. Cancer Res. 2007, 67:8285-8292; Janke et al., 2007, Gene Ther. 14:1639-1649). However, it should be noted that the replication capability of a recombinant NDV expressing a transgene may be hampered in comparison with its parental NDV strain not expressing the transgene. As discussed above, the viral F protein of NDV is responsible for viral fusion with the cell membrane and for viral spread from host cell to host cell via formation of syncytia. The presence of the multi-basic amino acid cleavage site in the F protein enables protein cleavage and activation by a broad range or proteases and is known to be a determinant of virulence in velogenic NDV strains. In order to increase the oncolytic potency of a highly attenuated lentogenic Hitchner B1 NDV strain, a polybasic cleavage site was introduced into the F protein to generate rNDV/F3aa with mutated F protein. Altomonte et al. (Mol Ther. 2010 18:275-84) reported an oncolytic NDV vector virus with the mutated F-protein rNDV/F(L289A), harboring an L289A mutation within the F protein, which is required for virus entry and cell-cell fusion. This rNDV/F3aa(L289A) virus with mutated F protein showed further enhanced fusion and cytotoxicity of hepatocellular carcinoma (HCC) cells in vitro, as compared with a rNDV/F3aa control virus. In vivo administration of rNDV/F3aa(L289A), via hepatic arterial infusion in immune-competent Buffalo rats bearing multifocal, orthotopic liver tumors resulted in tumor-specific syncytia formation and necrosis, with no evidence of toxicity to the neighboring hepatic parenchyma, which translated to a 20% prolongation of survival time relative to treatment with the original rNDV/F3aa virus.

Due to compelling preclinical data implicating oncolytic NDV as an ideal candidate for cancer therapy, phase I and II clinical trials have been initiated. Several trials have shown that NDV is a safe and potentially therapeutically useful therapeutic agent, with no reports of significant adverse effects in patients beyond conjunctivitis or mild flu-like symptoms (Csatary et al., 1999, Anticancer Res. 19:635-638; Pecora et al., 2002, J. Clin. Oncol. 20:2251-2266; Lorence et al., 2003, Curr. Opin. Mol. Ther. 5, 618-624; Freeman et al., 2006, Mol. Ther. 13:221-228). Although the results of these clinical trials have been encouraging, the extent of clinical responses has not been strong and robust enough to consider bringing one of these initial NDV strains into advanced clinical development. Thus, strategies for improving therapeutic effectiveness while maintaining an acceptable safety profile of oncolytic NDV strains are needed. Thus there remains a clear need for improvement in therapeutic outcome by providing improved NDV strains, in particular NDV strains with enhanced killing effect without affecting—or even improving—its safety toward normal cells.

### DESCRIPTION OF THE PRESENT INVENTION

In a first aspect the present invention provides recombinant Newcastle disease virus or NDV strains. A recombinant NDV according to the present invention carries as a foreign gene at least one gene selected from:
A gene encoding an antibody directed to the protein PD-L1 or an antigen-binding part directed to the protein PD-L1 (anti-PD-L1), such as Atezolizumab or a variant or an antigen-binding part thereof.
A gene encoding an antibody directed to the growth factor protein VEGF-A or an antigen-binding part directed to the growth factor protein VEGF-A (anti-VEGF-A), such as Bevacizumab or a variant or an antigen-binding part thereof.
A gene encoding an antibody directed to the killer-cell immunoglobulin-like receptors (KIRs), in particular KIR2DL1/2L3, thereby regulating the killing function of the cells expressing the receptors, or an antigen-binding part directed to killer-cell immunoglobulin-like receptors (KIRs), in particular KIR2DL1/2L3, (anti-KIR).
A gene encoding an antibody directed to and inhibiting LAG-3 or an antigen-binding part directed to directed to and inhibiting LAG-3 (anti-LAG-3). LAG-3 is a protein, which is present on the surface of T-cells. When the antibody or its antigen-binding part is bound to LAG-3 on T-cells, the T-cells are stimulated to attack cancer cells.
A gene encoding an antibody directed to NKG2A or an antigen-binding part directed to protein NKG2A (anti- NKG2A). Binding of the antibody to NKG2a prevents the binding of NKG2A to its ligand human leukocyte antigen-E (HLA-E), which induces an immune response against the cancer cells leading to their destruction.
A gene encoding an antibody directed to the glucocorticoid-induced TNF-superfamily receptor (GITR), thereby blocking the interaction of GITR with its ligand, or an antigen-binding part directed to the glucocorticoid-induced TNF-superfamily receptor (GITR) (anti-GITR).
A gene encoding an antibody directed to and activating the protein OX40 (also known as CD134 or TNFRSF4), thereby increasing the number of T-cells, or an antigen-binding part directed to protein OX40 (anti-OX40).
A gene encoding a membrane protein which is the receptor for the proteins CD28 and CTLA-4, and which membrane protein is involved in the costimulatory signal essential for T-lymphocyte activation, or a part thereof which is the receptor for the proteins CD28 and CTLA-4, and which membrane protein is involved in the costimulatory signal essential for T-lymphocyte activation.
A gene encoding a human interleukin 12 (hIL-12) protein, or a part thereof.
A gene encoding a green fluorescent protein, which can be used for the localization and monitoring of cells, in particular cancer cells, or a part thereof.
Any combination of these genes, parts or variants of these genes or parts thereof.

The foreign gene or part or the respective variant is/are expressed in the recombinant NDV strain of the present invention, when the virus is replicating in a suitable host or host cell. An expression of a gene means that the nucleic acid information encoded by the gene is translated in a respective amino acid sequence, which is the primary sequence for building up the respective protein or gene product. By way of example, the gene product of the HN gene is the HN protein.

Within the present invention the foreign gene carried by the recombinant NDV encodes in one embodiment an antibody or an antigen-binding part of the antibody directed to the protein PD-L1 (anti-PD-L1). According to the present invention the antibody is in a particularly preferred embodiment Atezolizumab, an antigen-binding part of Atezolizumab, a variant of Atezolizumab or a variant of an antigen-binding part of Atezolizumab.

According to another embodiment of the present invention the foreign gene carried by the recombinant NDV encodes an antibody or an antigen-binding part of the antibody directed to the growth factor protein VEGF-A (anti-VEGF-A). VEGF-A is a growth factor protein that stimulates angiogenesis in a variety of diseases, especially in cancer. Thus the antibody is capable of blocking angiogenesis. According to the present invention the antibody is in a particularly preferred embodiment Bevacizumab, an antigen-binding part of Bevacizumab, a variant of Bevacizumab or a variant of an antigen-binding part of Bevacizumab.

According to another embodiment of the present invention the foreign gene carried by the recombinant NDV encodes an antibody directed to killer-cell immunoglobulin-like receptors (KIRs), in particular KIR2DL1/2L3, or an antigen-binding part directed to killer-cell immunoglobulin-like receptors (KIRs), in particular KIR2DL1/2L3. According to the present invention the antibody or antigen-binding part thereof is in a particularly preferred embodiment Lirilumab, an antigen-binding part of Lirilumab, a variant of Lirilumab or a variant of an antigen-binding part of Lirilumab. They work by regulating the killing function of the cells expressing the receptors.

According to another embodiment of the present invention the foreign gene carried by the recombinant NDV encodes an antibody directed to and inhibiting LAG-3, a protein, which is present on the surface of T-cells, or an antigen-binding part directed to and inhibiting LAG-3. According to the present invention the antibody or antigen-binding part thereof is in a particularly preferred embodiment Relatlimab, an antigen-binding part of Relatlimab, a variant of Relatlimab or a variant of an antigen-binding part of Relatlimab. When bound to LAG-3 on T-cells, the T-cells are stimulated to attack cancer cells.

According to another embodiment of the present invention the foreign gene carried by the recombinant NDV encodes an antibody directed to NKG2A, or an antigen-binding part directed to NKG2A. According to the present invention the antibody or antigen-binding part thereof is in a particularly preferred embodiment Monalizumab, an antigen-binding part of Monalizumab, a variant of Monalizumab or a variant of an antigen-binding part of Monalizumab. Binding of the antibody or its antigen-binding part to NKG2A prevents the binding of NKG2A to its ligand human leukocyte antigen-E (HLA-E), which induces an immune response against the cancer cells leading to their destruction.

According to another embodiment of the present invention the foreign gene carried by the recombinant NDV encodes an antibody directed to the glucocorticoid-induced TNF-superfamily receptor (GITR), or an antigen-binding part directed to the glucocorticoid-induced TNF-superfamily receptor (GITR). According to the present invention the antibody or antigen-binding part thereof is in a particularly preferred embodiment TRX518, an antigen-binding part of TRX518, a variant of TRX518 or a variant of an antigen-binding part of TRX518. The antibody or its antigen-binding part acts by blocking the interaction of GITR with its ligand.

According to another embodiment of the present invention the foreign gene carried by the recombinant NDV encodes an antibody directed to and activating the protein OX40, or an antigen-binding part directed to and activating the protein OX40. According to the present invention the antibody or antigen-binding part thereof is in a particularly preferred embodiment BMS 986178, an antigen-binding part of BMS 986178, a variant of BMS 986178 or a variant of an antigen-binding part of BMS 986178. The antibody or its antigen-binding part is capable of increasing the number of T-cells.

According to another embodiment of the present invention the foreign gene carried by the recombinant NDV encodes a membrane protein which is the receptor for the proteins CD28 and CTLA-4, and which membrane protein is involved in the costimulatory signal essential for T-lymphocyte activation, or a part thereof which is the receptor for the proteins CD28 and CTLA-4, and which membrane protein is involved in the costimulatory signal essential for T-lymphocyte activation. According to the present invention the membrane protein or part thereof is in a particularly preferred embodiment CD80 (cluster of differentiation 80), a part of CD80, a variant of CD80 or a variant of a part of CD80.

According to another embodiment of the present invention the foreign gene carried by the recombinant NDV encodes a human interleukin 12 (hIL-12), or a part thereof. According to the present invention the protein or part thereof is in a particularly preferred embodiment (ns)hIL-12, a part of (ns)hIL-12, a variant of (ns)hIL-12 or a variant of a part of (ns)hIL-12. As used herein “(ns)” is intended to mean “non secreting”. That is the interleukin is preferred to be a non secreting human interleukin 12. A non secreting interleukin may be advantageous over a normal interleukin, as it may prevent that there is an uncontrolled immune response, for example in the form of a massive cytokine secretion, triggered by administration of the recombinant NDV strain.

According to another embodiment of the present invention the foreign gene carried by the recombinant NDV encodes a green fluorescent protein, which can be used for the localization and monitoring of cells, in particular cancer cells, or a part thereof. According to the present invention the protein or part thereof is in a particularly preferred embodiment enhanced green fluorescent protein (eGFP), a part of enhanced green fluorescent protein (eGFP), a variant of enhanced green fluorescent protein (eGFP) or a variant of a part of eGFP.

In another embodiment of the present invention a recombinant NDV comprises in its viral genome at least two foreign genes or parts or variants thereof, which foreign genes are selected from any one of the above mentioned foreign genes. In one embodiment the foreign genes are selected from a gene encoding an antibody or an antigen-binding part of the antibody directed to the protein PD-L1 or a part thereof, preferably Atezolizumab, an antigen-binding part thereof or a variant of Atezolizumab or its antigen-binding part, and a gene encoding an antibody or an antigen-binding part of the antibody directed to the growth factor protein VEGF-A or a part thereof, preferably Bevacizumab, an antigen-binding part thereof or a variant of Bevacizumab or its antigen-binding part.

The above mentioned group of foreign genes will also be referred to the following as “the foreign genes of the present invention”. This term is intended to also include any parts of these genes as well as the particularly preferred foreign genes (encoding Atezolizumab, Bevacizumab, Lirilumab, Relatlimab, Monalizumab, TRX518, BMS 986178, CD80, (ns)hIL-12 or eGFP) and parts or variants thereof. Even though in the following the present invention will be described in particular with reference to the foreign genes encoding Atezolizumab or Bevacizumab, it should be understood that the respective teaching and all disclosed preferred embodiments of such Atezolizumab or Bevacizumab carrying NDV strains also apply to the other foreign genes, parts or variants.

A “variant” of a starting nucleic acid is a nucleic acid that comprises a nucleic acid sequence different from that of the starting nucleic acid. Generally, a variant will possess at least 75% sequence identity, preferably at least 90% sequence identity, more preferably at least 95% sequence identity, and most preferably at least 98% sequence identity with the native nucleic acid. Variants of a nucleic acid may be prepared by introducing appropriate nucleotide changes into the nucleic acid. Such variants include, for example, deletions from, and/or insertions into and/or substitutions of residues within the nucleic acid sequence of the gene of interest. Any combination of deletion, insertion, and substitution is possibly made to arrive at the final construct, provided that the final construct possesses the desired characteristics. Methods for generating nucleic acid sequence variants are known to the skilled person.

A “variant” of a starting polypeptide or protein, e.g. an antibody or another protein, is a polypeptide or protein that comprises an amino acid sequence different from that of the starting polypeptide or protein. Generally, a variant will possess at least 75% sequence identity, preferably at least 90% sequence identity, more preferably at least 95% sequence identity, and most preferably at least 98% sequence identity with the native polypeptide or protein. Variants of a polypeptide or a protein may be prepared by introducing appropriate nucleotide changes into the nucleic acid encoding the polypeptide or protein. Such variants include, for example, deletions from, and/or insertions into and/or substitutions of residues within the amino acid sequence of the polypeptide/protein of interest. Any combination of deletion, insertion, and substitution is possibly made to arrive at the final construct, provided that the final construct possesses the desired characteristics. The amino acid changes also may alter post-translational processes of the polypeptide, such as changing the number or position of glycosylation sites. Methods for generating amino acid sequence variants of polypeptides are known to the skilled person.

Percentage sequence identity is determined after the best alignment of the sequence of interest with the respective reference sequence. The best alignment of the sequences may for example be produced by means of the Fitchet al., Proc. Natl. Acad. Sci. USA 80:1382-1386 (1983), version of the algorithm described by Needleman et al., J. Mol. Biol. 48:443-453 (1970), after aligning the sequences to provide for best homology, by means of the similarity search method of Pearson and Lipman, Proc. Natl Acad. Sci. USA 85, 2444 (1988), or by means of computer programs which use these algorithms (in particular BLASTN in Wisconsin Genetics Software Package, Genetics Computer Group, 575 Science Drive, Madison, Wis.). The percentage identity is calculated by determining the number of identical positions between the two sequences being compared, dividing this number by the number of positions compared and multiplying the result obtained by 100 so as to obtain the percentage identity between these two sequences. Preferably, sequence identity of a nucleotide sequence is determined using BLASTN, preferably BLASTN in standard settings as provided by the website of the U.S. National Library of Medicine “https://blast.ncbi.nlm.nih.gov”. Also preferably, sequence identity of an amino acid sequence is determined using BLASTP, preferably BLASTP in standard settings as provided by the website of the U.S. National Library of Medicine “https://blast.ncbi.nlm.nih.gov”. It is further preferred that the sequence identity is calculated over the entire length of the respective sequence.

As used herein, the term “part” means a fragment of a starting nucleic acid or amino acid sequence, which fragment is a made up of a consecutive sequence of the nucleotides or amino acids of the starting sequence. The part or fragment preferably comprises at least 10, more preferably at least 20 and preferably up to 500 nucleotides or amino acids. In any case a part according to the present invention is a functional part. That is nucleic acid or protein encoded by that nucleic acid must provide the desired function, e.g. an antigen-binding function or expression inhibiting function as specified herein, of the starting gene or protein.

In addition to carrying at least one foreign gene or a part or variant thereof, the recombinant NDV strains according to the present invention further comprise a mutation in the viral HN gene leading to a change in the amino acid sequence of the HN protein and providing the NDV with an increased replication capability in a cancer cell, preferably in a human cancer cell, as compared to an otherwise identical NDV not having the said mutation in the HN gene.

Preferably the mutation in the HN gene results in an unexpected beneficially increased replication capability of the recombinant NDV strain in cancer cells, preferably in human cancer cells, and more preferably results in a replication of said oncolytic NDV in a human cancer cell which is at least 2-fold higher, more preferably 2- to 10-fold higher, still more preferably 5- to 10-fold higher than an NDV parent strain not having said mutation in its viral HN gene. Preferably, said parent strain comprises or is an oncolytic NDV already having an advantageous safety and efficacy profile in humans, such as the NDV strain MTH-68/H.

According to a preferred embodiment the recombinant NDV strain comprises a mutated HN gene and the encoded hemagglutinin-neuramidase (HN protein) with an amino acid substitution at position 277 to an amino acid with a hydrophobic side chain other than phenylalanine. Still more preferred is the amino acid phenylalanine (F) substituted to leucine (L) at amino acid position 277 of the HN gene. In a more preferred embodiment, the invention provides an oncolytic NDV derived from NDV strain MTH-68/H having this mutation in the HN gene and carrying at least one of the foreign genes or parts or functional analogs thereof as specified supra, said oncolytic NDV according to the invention having an unexpected beneficially increased replication capability allowing replication of said oncolytic NDV in a human cancer cell preferably up to at least 10-fold higher levels than the oncolytic NDV parent strain MTH-68/H, thereby maintaining the useful oncological safety and efficacy specifications of the parent virus and advantageously supplementing it with the increased replication capability of an oncolytic NDV and the ability to express within infected cancer cells at least one of the before mentioned foreign genes, which beneficially influence the immunological response towards the cancer cells. Herein an oncolytic NDV having such a mutation is also identified as NDV^{F277L} to discern it from a parent NDV having a phenylalanine at amino acid position 277 of the HN gene.

A NDV strain derived from NDV strain MTH-68/H having a mutation in the HN gene product or HN protein at position 277, wherein the amino acid phenylalanine (F) is substituted to leucine (L) at position 277 is referred to herein also as NDV-Mut HN(F277L). The foreign gene or part or functional analog thereof carried by this strain is referred to as Atezolizumab, Bevacizumab, Lirilumab, Relatlimab, Monalizumab, TRX518, BMS 986178, CD80, (ns)hIL-12, or eGFP in this shortcut for the strain.

Within the shortcut for the recombinant NDV strains as used herein a reference to e.g. Atezolizumab is intended to comprise the complete antibody, antigen-binding parts, and variants of the antibody or of antigen-binding parts, but also encompasses the more general form of an anti-PD-L1-antibody and antigen-binding parts of this antibody. A reference to Bevacizumab is again intended to comprise the complete antibody, antigen-binding parts and variants of the antibody or antigen-binding parts, but also the more general form of an antibody to the growth factor protein VEGF-A, and antigen-binding parts of this antibody. A reference to Lirilumab is intended to comprise the complete antibody, antigen-binding parts, and variants of the antibody or of antigen-binding parts, but also encompasses the more general form of an anti-MR-antibody and antigen-binding parts of this antibody. A reference to Relatlimab is intended to comprise the complete antibody, antigen-binding parts, and variants of the antibody or of antigen-binding parts, but also encompasses the more general form of an anti-LAG-3-antibody and antigen-binding parts of this antibody. A reference to Monalizumab is intended to comprise the complete antibody, antigen-binding parts, and variants of the antibody or of antigen-binding parts, but also encompasses the more general form of an anti-NKG2A-antibody and antigen-binding parts of this antibody. A reference to TRX518 is intended to comprise the complete antibody, antigen-binding parts, and variants of the antibody or of antigen-binding parts, but also encompasses the more general form of an anti-GITR-antibody and antigen-binding parts of this antibody. A reference to BMS 986178 is intended to comprise the complete antibody, antigen-binding parts, and variants of the antibody or of antigen-binding parts, but also encompasses the more general form of an anti-OX40-antibody and antigen-binding parts of this antibody.

A reference to CD80 is intended to comprise the complete protein, parts and variants of the protein, as well as the more general form of a membrane protein which is the receptor for the proteins CD28 and CTLA-4, and which membrane protein is involved in the costimulatory signal essential for T-lymphocyte activation, or a respective part thereof. A reference to (ns)hIL-12 is intended to comprise the complete protein, parts and variants of the protein, as well as the more general form of a human interleukin 12 (hIL-12), or a respective part thereof. A reference to eGFP is intended to comprise the complete protein, parts and variants of the protein, as well as the more general form of a green fluorescent protein, or a respective part thereof.

That is a recombinant NDV strain referred to as “NDV-Mut HN(F277L)-Atezolizumab” is a NDV strain derived from NDV strain MTH-68/H having a mutation in the HN gene resulting in an amino acid exchange at position 277, wherein the amino acid phenylalanine (F) is substituted to leucine (L) at position 277, and carrying as a foreign gene a gene encoding an antibody that is directed to the protein PD-L1, preferably Atezolizumab or a variant thereof. A recombinant NDV strain referred to as “NDV-Mut HN(F277L)-Bevacizumab” is a NDV strain derived from NDV strain MTH-68/H having a mutation in the HN gene resulting in an amino acid exchange at position 277, wherein the amino acid phenylalanine (F) is substituted to leucine (L) at position 277, and carrying as a foreign gene a gene encoding an antibody that is directed to the growth factor protein VEGF-A, preferably Bevacizumab or a variant thereof. The NDV strains NDV-Mut HN(F277L)-Atezolizumab and NDV-Mut HN(F277L)-Bevacizumab are provided by the present invention in a preferred embodiment. Beside these both NDV-strains the following NDV strains are provided by the present invention in a preferred embodiment:
NDV-Mut HN(F277L)-Lirilumab
NDV-Mut HN(F277L)-Relatlimab
NDV-Mut HN(F277L)-Monalizumab
NDV-Mut HN(F277L)-TRX518
NDV-Mut HN(F277L)-BMS 986178
NDV-Mut HN(F277L)-CD80
NDV-Mut HN(F277L)-(ns)hIL-12
NDV-Mut HN(F277L)-eGFP

Within the meaning of the present invention the term “Newcastle disease virus” or “NDV” includes all known velogenic, mesogenic and lentogenic NDV strains. According to the present invention mesogenic and lentogenic strains are preferred. Still more preferred is the NDV derived from NDV strain MTH-68/H.

Within the meaning of the present invention “derived from NDV strain MTH-68/H” is intended to mean that the derived NDV strain is encoded by a viral genome or comprises a viral genome having a nucleic acid sequence with a sequence identity of at least 75%, particularly at least 90%, more particularly at least 95% to the nucleic acid sequence of the NDV strain MTH-68/H. In one embodiment the sequence identity is at least 99% or 100%. Any foreign genes comprised in the recombinant NDV strains are not considered in the sequence alignment, and the reference sequence for the sequence alignment is that of SEQ ID No. 1 of the sequence listing. A sequence variation is for example the substitution, insertion or deletion of at least one nucleotide, preferably the substitution, insertion or deletion of up to 20 nucleotides, still more preferably of up to 15 nucleotides. Any combination of substitution, insertion and deletion is also comprised by that definition, provided that the final construct possesses the desired characteristics. The variation may result in at least one amino acid substitution, wherein each amino acid substitution can be a conservative or a non-conservative amino acid substitution.

It has been surprisingly shown that the mutation in the HN gene results in a beneficially improved replication capability of the respective strain as compared to an otherwise identical NDV not having said mutation in the HN gene. The beneficially increased replication rate of the NDV not only results in higher number of virus particles capable of infecting and destroying cancer cells, but also results in a higher expression of the foreign gene carried by the said recombinant NDV strains. As explained earlier, the respective gene products of the foreign gene results in an improved immunological response and thus an improved destruction of cancer cells.

In a preferred embodiment of the present invention the recombinant and mutated NDV strains comprise in addition to the above described mutation in the HN gene, a mutation in the M gene and the thus encoded matrix protein. Still more preferred the mutated M gene encodes a matrix protein with an amino acid substitution at position 165 to an amino acid with an aromatic side chain, wherein still more preferred the amino acid glycine (G) at amino acid position 165 of the M gene is substituted to the amino acid tryptophane (W).

NDV strains comprising both a mutation in the HN gene and the M gene, as described supra, have been shown as being particularly suitable to infect cancer cells and to provide an enhanced replication rate within the cancer cells. Thus, according to one embodiment of the present invention a recombinant NDV strain comprises as a foreign gene at least one gene selected from the group of the foreign genes of the present invention, as defined above. In one embodiment of the present invention a recombinant NDV strain comprises as a foreign gene at least one a gene encoding an antibody directed to the protein PD-L1 or a part thereof, preferably Atezolizumab, a part thereof or a variant of Atezolizumab or its part, a gene encoding an antibody directed to the growth factor protein VEGF-A or a part thereof, preferably Bevacizumab, a part thereof or a variant of Bevacizumab or its part, and any combination of these genes or variants thereof, and has a mutation both in the HN gene, preferably resulting in a substitution of the amino acid phenylalanine (F) to leucine (L) at position 277 of the HN gene product, and a mutation in the M gene, preferably resulting in a substitution of the amino acid glycine (G) to the amino acid tryptophane (W) at position 165 of the M gene product. As explained supra, other substitutions at these two amino acid positions are also comprised within the present invention.

A NDV strain derived from NDV strain MTH-68/H having a mutation in the HN gene, wherein the amino acid phenylalanine (F) is substituted to leucine (L) at position 277 of the HN gene product, and having a mutation in the M gene, wherein the amino acid glycine (G) is substituted to the amino acid tryptophane (W) at position 165 of the M gene product is herein referred to also as NDV-Mut HN(F277L)/M(G165W) or MutHu or NDV-NoThaBene-1. The foreign gene or part or variant thereof carried by this strain is referred to as Atezolizumab, Bevacizumab, Lirilumab, Relatlimab, Monalizumab, TRX518, BMS 986178, CD80, (ns)hIL-12 or eGFP. Within these shortcuts for the recombinant NDV strains a reference to the respective foreign gene is intended to have the meaning as already defined above. The following NDV strains are provided by the present invention in a particularly preferred embodiment:
NDV-Mut HN(F277L)/M(G165W)-Atezolizumab,
NDV-Mut HN(F277L)/M(G165W)-Bevacizumab,
NDV-Mut HN(F277L)/M(G165W)-Lirilumab,
NDV-Mut HN(F277L)/M(G165W)-Relatlimab,
NDV-Mut HN(F277L)/M(G165W)-Monalizumab,
NDV-Mut HN(F277L)/M(G165W)-TRX518,
NDV-Mut HN(F277L)/M(G165W)-BMS 986178,
NDV-Mut HN(F277L)/M(G165W)-CD80,
NDV-Mut HN(F277L)/M(G165W)-(ns)hIL-12,
NDV-Mut HN(F277L)/M(G165W)-eGFP.

A recombinant NDV strain referred to as “NDV-Mut HN(F277L)/M(G165W)-Atezolizumab” is a NDV strain derived from NDV strain MTH-68/H having a mutation in the HN gene at amino acid position 277, wherein the amino acid phenylalanine (F) is substituted to leucine (L) at position 277, and having a mutation in the M gene, wherein the amino acid glycine (G) is substituted to the amino acid tryptophane (W) at position 165 of the M gene product, and carrying as a foreign gene a gene encoding an antibody that is directed to the protein PD-L1, preferably Atezolizumab, or a variant thereof. A recombinant NDV strain referred to as “NDV-Mut HN(F277L)/M(G165W)-Bevacizumab” is a NDV strain derived from NDV strain MTH-68/H having a mutation in the HN gene at amino acid position 277, wherein the amino acid phenylalanine (F) is substituted to leucine (L) at amino acid position 277, and having a mutation in the M gene, wherein the amino acid glycine (G) is substituted to the amino acid tryptophane (W) at position 165 of the M gene product, and carrying as a foreign gene a gene encoding an antibody directed to the growth factor protein VEGF-A or a part thereof, preferably Bevacizumab or a variant thereof. The NDV strains NDV-Mut HN(F277L)/M(G165W)-Atezolizumab and NDV-Mut HN(F277L)/M(G165W)-Bevacizumab are provided by the present invention in a preferred embodiment.

Optionally the recombinant and mutated NDV strains may comprise in addition to the above described mutation in the HN gene, at least one mutation in the F gene and the thus encoded fusion protein. Still more preferred the mutated F gene encodes a fusion protein with an amino acid substitution at amino acid position 117 to an amino acid with a hydroxylated side chain, preferably where phenylalanine (F) is substituted to serine (S) at position 117 of the F gene product, and/or with an amino acid substitution at amino acid position 190 to an amino acid with an aliphatic amino acid side chain, preferably where phenylalanine (F) is substituted to leucine (L) at position 190 of the F gene product. The combination of both mutations at amino acid position 117 and 190 of the F gene is particularly preferred. These recombinant NDV strains may in addition also comprise the above described mutation in the M gene.

Thus, according to one embodiment of the present invention a recombinant NDV strain comprises as a foreign gene at least one gene selected from the group of the foreign genes of the present invention as defined. In one embodiment of the present invention there is provided a recombinant NDV strain comprises as a foreign gene a gene encoding an antibody directed to the protein PD-L1 or a part thereof, preferably Atezolizumab, an antigen-binding part thereof or a variant of Atezolizumab or its antigen-binding part, a gene encoding an antibody directed to the growth factor protein VEGF-A or a part thereof, preferably Bevacizumab, an antigen-binding part thereof or a variant of Bevacizumab or its antigen-binding part, and any combination of these genes or parts or variants thereof, and has mutations both in the HN gene, preferably resulting in a substitution of the amino acid phenylalanine (F) to leucine (L) at position 277 of the HN gene product, and optionally may further have at least one mutation in the F gene, preferably resulting in a substitution of the amino acid phenylalanine (F) to the amino acid serine (S) at position 117 of the F gene product, and/or resulting in a substitution of the amino acid phenylalanine (F) to the amino acid leucine (L) at position 190 of the F gene product. The combination of both mutations at amino acid position 117 and 190 of the F gene is particularly preferred. These recombinant NDV strains may in addition also comprise the above described mutation in the M gene, preferably resulting in a substitution of the amino acid glycine (G) to the amino acid tryptophane (W) at amino acid position 165 of the M gene. As explained supra, other substitutions at these amino acid positions are also comprised within the present invention.

Optionally, the recombinant and mutated NDV strains may comprise in addition to the above described mutation in the HN gene, at least one mutation in the L gene and the thus encoded RNA-dependent RNA polymerase protein, also referred to as L protein or L gene product herein. Preferably the mutated L gene encodes a RNA-dependent RNA polymerase protein with an amino acid substitution at position 757 to an amino acid with an aliphatic amino acid side chain other than valine (V), preferably where valine (V) is substituted to isoleucine (I) at position 757 of the L gene product, and/or with an amino acid substitution at position 1551 to an amino acid with a hydroxylated side chain, preferably where phenylalanine (F) is substituted to serine (S) at position 1551 of the L gene product, and/or with an amino acid substitution at position 1700 to an amino acid with an aliphatic side chain, preferably where arginine (R) is substituted to leucine (L) at position 1700 of the L gene product, preferably wherein the NDV comprises a mutated L gene and the encoded L protein with the amino acid substitution at position 757, position 1551 and position 1700. These recombinant NDV strains may in addition also comprise the above described mutation in the M gene and/or one or more of the above described mutations in the F gene.

Thus, according to one embodiment of the present invention a recombinant NDV strain comprises as a foreign gene at least one gene selected from the group of the foreign genes of the present invention as defined above. Optionally, a recombinant NDV strain may comprise as a foreign gene at least one gene selected from a gene encoding an antibody directed to the protein PD-L1 or a part thereof, preferably Atezolizumab or an antigen-binding part thereof or a variant of Atezolizumab or its antigen-binding part, a gene encoding an antibody directed to the growth factor protein VEGF-A or a part thereof, preferably Bevacizumab, an antigen-binding part thereof or a variant of Bevacizumab or its antigen-binding part, and any combination of these genes or parts or variants thereof, and has mutations both in the HN gene, preferably resulting in a substitution of the amino acid phenylalanine (F) to leucine (L) at amino acid position 277 of the HN gene, and at least one mutation in the L gene, preferably one resulting at amino acid position 757 to an amino acid with an aliphatic amino acid side chain other than valine (V), preferably where valine (V) is substituted to isoleucine (I) at position 757 of the L gene product, and/or with an amino acid substitution at position 1551 to an amino acid with a hydroxylated side chain, preferably where phenylalanine (F) is substituted to serine (S) at position 1551 of the L gene product, and/or with an amino acid substitution at position 1700 to an amino acid with an aliphatic side chain, preferably where arginine (R) is substituted to leucine (L) at position 1700 of the L gene product, preferably wherein the NDV comprises a mutated L gene and the encoded L protein with the amino acid substitution at position 757, position 1551 and position 1700. These recombinant NDV strains may in addition also comprise the above described mutation in the M gene, preferably resulting in a substitution of the amino acid glycine (G) to the amino acid tryptophane (W) at position 165 of the M gene product, and/or one or more of the above described mutations in the F gene, preferably resulting in a substitution of the amino acid phenylalanine (F) to the amino acid serine (S) at position 117 of the gene product and/or resulting in a substitution of the amino acid phenylalanine (F) to the amino acid leucine (L) at position 190 of the F gene product. As explained supra, other substitutions at these amino acid positions are also comprised within the present invention.

A NDV strain derived from NDV strain MTH-68/H having a mutation in the HN gene, wherein the amino acid phenylalanine (F) is substituted to leucine (L) at amino acid position 277, having a mutation in the M gene, wherein the amino acid glycine (G) is substituted to the amino acid tryptophane (W) at position 165 of the M gene product, optionally having two mutations in the F gene, wherein the amino acid phenylalanine (F) is substituted to the amino acid serine (W) at position 117 and the amino acid phenylalanine (F) is substituted to the amino acid leucine (L) at position 190 of the F gene product, and optionally having three mutations in the L gene, wherein the amino acid valine (V) is substituted to the amino acid isoleucine (I) at position 757, the amino acid phenylalanine (F) is substituted to the amino acid serine (S) at position 1551, and the amino acid arginine (R) is substituted to the amino acid leucine (L) at position 1700 of the L gene product is herein referred to also as NDV-Mut HN(F277L)/M(G165W)/F(F117S)/F(F190L)/L(V757I)/L(F1551S)/L(R1700L) or MutHu2 or NDV-NoThaBene-2. The foreign gene or part or variant thereof carried by this strain is referred to as Atezolizumab, Bevacizumab, Lirilumab, Relatlimab, Monalizumab, TRX518, BMS 986178, CD80, (ns)hIL-12 or eGFP. Within these shortcuts for the recombinant NDV strains a reference to the respective foreign gene is intended to have the meaning as already defined above. Optionally the following NDV strains may further be provided:
NDV-Mut HN(F277L)/M(G165W)/F(F117S)/F(F190L)/L(V757I)/L(F1551S)/L(R1700L)-Atezolizumab,
NDV-Mut HN(F277L)/M(G165W)/F(F117S)/F(F190L)/L(V757I)/L(F1551S)/L(R1700L)-Bevacizumab,
NDV-Mut HN(F277L)/M(G165W)/F(F117S)/F(F190L)/L(V757I)/L(F1551S)/L(R1700L)-Lirilumab,
NDV-Mut HN(F277L)/M(G165W)/F(F117S)/F(F190L)/L(V757I)/L(F1551S)/L(R1700L)-Relatlimab,
NDV-Mut HN(F277L)/M(G165W)/F(F117S)/F(F190L)/L(V757I)/L(F1551S)/L(R1700L)-Monalizumab,
NDV-Mut HN(F277L)/M(G165W)/F(F117S)/F(F190L)/L(V757I)/L(F1551S)/L(R1700L)-TRX518,
NDV-Mut HN(F277L)/M(G165W)/F(F117S)/F(F190L)/L(V757I)/L(F1551S)/L(R1700L)-BMS 986178,
NDV-Mut HN(F277L)/M(G165W)/F(F117S)/F(F190L)/L(V757I)/L(F1551S)/L(R1700L)-CD80,
NDV-Mut HN(F277L)/M(G165W)/F(F117S)/F(F190L)/L(V757I)/L(F1551S)/L(R1700L)-(ns)hIL-12,
NDV-Mut HN(F277L)/M(G165W)/F(F117S)/F(F190L)/L(V757I)/L(F1551S)/L(R1700L)-eGFP.

Optionally, a further recombinant NDV strain referred to as “NDV-Mut HN(F277L)/M(G165W)/F(F117S)/F(F190L)/L(V757I)/L(F1551S)/L(R1700L)-Atezolizumab” is a NDV strain derived from NDV strain MTH-68/H having a mutation in the HN gene product at position 277, wherein the amino acid phenylalanine (F) is substituted to leucine (L) at position 277, and having a mutation in the M gene, wherein the amino acid glycine (G) is substituted to the amino acid tryptophane (W) at position 165 of the M gene product, having two mutations in the F gene, wherein the amino acid phenylalanine (F) is substituted to the amino acid serine (S) at position 117 and the amino acid phenylalanine (F) is substituted to the amino acid leucine (L) at position 190 of the F gene product, and having three mutations in the L gene, wherein the amino acid valine (V) is substituted to the amino acid isoleucine (I) at position 757, the amino acid phenylalanine (F) is substituted to the amino acid serine (S) at position 1551, and the amino acid arginine (R) is substituted to the amino acid leucine (L) at position 1700 of the L gene product, and carrying as a foreign gene a gene encoding an antibody or an antigen-binding part directed to the protein PD-L1, preferably Atezolizumab or an antigen-binding part thereof or a variant of Atezolizumab or an antigen-binding part of Atezolizumab. Optionally, a further recombinant NDV strain referred to as “NDV-Mut HN(F277L)/M(G165W)/F(F117S)/F(F190L)/L(V757I)/L(F1551S)/L(R1700L)-Bevacizumab” is a NDV strain derived from NDV strain MTH-68/H having a mutation in the HN gene at amino acid position 277 of the respective gene product, wherein the amino acid phenylalanine (F) is substituted to leucine (L) at position 277, having a mutation in the M gene, wherein the amino acid glycine (G) is substituted to the amino acid tryptophane (W) at position 165 of the M gene product, having two mutations in the F gene, wherein the amino acid phenylalanine (F) is substituted to the amino acid serine (S) at position 117 and the amino acid phenylalanine (F) is substituted to the amino acid leucine (L) at position 190 of the F gene product, and having three mutations in the L gene, wherein the amino acid valine (V) is substituted to the amino acid isoleucine (I) at position 757, the amino acid phenylalanine (F) is substituted to the amino acid serine (S) at position 1551, and the amino acid arginine (R) is substituted to the amino acid leucine (L) at position 1700 of the L gene product, and carrying as a foreign gene a gene encoding an antibody or an antigen-binding part thereof directed to the growth factor protein VEGF-A, preferably Bevacizumab or an antigen-binding part thereof or a variant of Bevacizumab or a variant of an antigen-binding part of Bevacizumab. The NDV strains NDV-Mut HN(F277L)/M(G165W)/F(F117S)/F(F190L)/L(V757I)/L(F1551S)/L(R1700L)-Atezolizumab and NDV-Mut HN(F277L)/M(G165W)/F(F117S)/F(F190L)/L(V757I)/L(F1551S)/L(R1700L)-Bevacizumab are optionally provided.

The two mutations in the F gene at position 117 and 190 of the amino acid sequence of the resulting F protein and the three mutations in the L gene at position 757, 1551 and 1700 of the amino acid sequence of the resulting L protein, either alone or in combination, have been shown to beneficially improve the oncolytic potential of the respective NDV strains by improving the safety profile of the viruses in humans. The combinations include any combination of the referenced mutations in the F gene and L gene, and the F protein and L protein, respectively. That is any combination from two to five mutations. Particularly preferred is a combination of both mutations at amino acid position 117 and 190 of the F gene (also referred to as “F2” herein) and a combination of all five mutations, that is mutations at positions 117 and 190 of the F gene product and at positions 757, 1551 and 1700 of the L gene product (also referred to as “F2L3” herein). The respective amino acid substitutions are as specified supra.

With regard to the viral safety profile, this in particular means that the intracerebral pathogenicity index (ICPI) is reduced by these mutations in the F gene, preferably F2, and/or the L gene, preferably F2L3. The ICPI value indicates the pathogenicity of a Newcastle disease virus. Preferably, the ICPI is reduced by at least 10%, more preferably by at least 20% and still more preferred by at least 30%, as compared to the identically determined ICPI value of a NDV strain not having the respective mutations in the F gene and/or the L gene. The ICPI can be determined according to the method set forth in World Organization for Animal Health (OIE) 2009, Chapter 2.3.14. Newcastle disease. pp. 576-589. In: Manual of Diagnostic Tests and Vaccines for Terrestrial Animals 2009. World Organization for Animal Health (OIE), Paris.

Optionally, in addition to the mutation at position 277 of the HN gene product and preferably one or more of the above specified mutations at position 165 of the M gene product, at positions 117 and/or 190 of F gene product, and at positions 757 and/or 1551 and/or 1700 of the L gene product, a recombinant NDV may also comprise a further mutation in the F gene, which mutation is a mutation which encodes a fusion protein having an amino acid substitution at position 289, and which mutation is capable of improving the oncolytic potential of the NDV strain. Still more preferred, the amino acid at position 289 of the F gene product is substituted from leucine (L) to alanine (A). Alternatively or in addition to the mutation at position 289 of the F protein, a recombinant NDV may optionally also comprise at least one further mutation in the L gene and the encoded L protein having an amino acid substitution at position 1717 to an amino acid with aromatic side chain other than tyrosine (Y), preferably where tyrosine (Y) is substituted to histidine (H) at position 1717 of the L gene product, and/or at position 1910 to an amino acid with a basic side chain, preferably where glutamic acid (E) is substituted to lysine (K) at position 1910 of the L gene product.

Alternatively or in addition a recombinant NDV according to the present invention may comprise a mutation in the RNA-editing sequence of the P gene. More preferably, the mutation abolishes and/or decreases the expression of the V protein.

Table 1a shows a list of mutations for obtaining NDV variants with increased titer production and/or improved safety profile according to the present invention. The mutation in the HN gene is comprised in all NDV strains according to the present invention, while the mentioned mutations in the M gene, F gene and L gene may or may not be present.

TABLE 1a

| Gene | Relevant amino acid |
|---|---|
| HN | F277L |
| M | G165W |
| F | F117S |
| F | F190L |
| F | L289A |
| L | V757I |
| L | F1551S |
| L | R1700L |
| L | Y1717H |
| L | E1910K |

Table 1b shows particularly preferred NDV variants with increased titer production and/or improved safety profile, and the parent NDV strain MTH-68/H. “+” means that at the mentioned amino acid position the original (first mentioned) amino acid is substituted by the last mentioned amino acid, while “−” means that there is no amino acid substitution at the mentioned position, i.e. the first mentioned amino acid is present. For example and with regard to the HN gene “+” means that at amino acid position 277 phenylalanine (F) is substituted to leucine (L).

TABLE 1b

| | Relevant | MTH- | | | | | | | | | | | | |
|---|---|---|---|---|---|---|---|---|---|---|---|---|---|---|
| Gene | amino acid | 68/H | | | | | | | | | | | | |
| HN | F277L | − | + | + | + | + | + | + | + | + | + | + | + | + |
| M | G165W | − | − | + | − | + | − | + | − | + | − | + | − | + |
| F | F117S | − | − | − | − | − | + | + | + | + | + | + | + | + |
| F | F190L | − | − | − | − | − | + | + | + | + | + | + | + | + |
| F | L289A | − | − | − | + | + | − | − | + | + | − | − | + | + |
| L | V757I | − | − | − | − | − | − | − | − | − | + | + | + | + |
| L | F1551S | − | − | − | − | − | − | − | − | − | + | + | + | + |
| L | R1700L | − | − | − | − | − | − | − | − | − | + | + | + | + |
| L | Y1717H | − | − | − | − | − | − | − | − | − | − | − | + | + |
| L | E1910K | − | − | − | − | − | − | − | − | − | − | − | + | + |

Table 1c shows all optional mutations in the F gene. “+” means that at the mentioned amino acid position the original (first mentioned) amino acid is substituted by the last mentioned amino acid, while “−” means that there is no amino acid substitution at the mentioned position, i.e. the first mentioned amino acid is present.

TABLE 1c

| | Relevant | | | | | | | |
|---|---|---|---|---|---|---|---|---|
| | amino | | | | | | | |
| Gene | acid | 1 | 1 | 1 | 2 | 2 | 2 | 3 |
| F | F117S | + | − | − | + | + | − | + |
| F | F190L | − | + | − | + | − | + | + |
| F | L289A | − | − | + | − | + | + | + |

Table 1d shows optional mutations in the L gene. “+” means that at the mentioned amino acid position the original (first mentioned) amino acid is substituted by the last mentioned amino acid, while “−” means that there is no amino acid substitution at the mentioned position, i.e. the first mentioned amino acid is present.

TABLE 1d

| | Relevant | | | | | | | | | | | | | | | | | | | | | |
|---|---|---|---|---|---|---|---|---|---|---|---|---|---|---|---|---|---|---|---|---|---|---|
| | amino | | | | | | | | | | | | | | | | | | | | | |
| Gene | acid | 1 | 1 | 1 | 2 | 2 | 2 | 3 | 2 | 2 | 3 | 3 | 3 | 3 | 4 | 3 | 3 | 4 | 4 | 4 | 4 | 5 |
| L | V757I | + | − | − | + | + | − | + | + | − | − | + | + | − | + | + | − | − | + | + | − | + |
| L | F1551S | − | + | − | + | − | + | + | − | + | − | + | − | + | + | − | + | − | + | − | + | + |
| L | R1700L | − | − | + | − | + | + | + | − | − | + | − | + | + | + | − | − | + | − | + | + | + |
| L | Y1717H | − | − | − | − | − | − | − | + | + | + | + | + | + | + | + | + | + | + | + | + | + |
| L | E1910K | − | − | − | − | − | − | − | − | − | − | − | − | − | − | + | + | + | + | + | + | + |

The NDV variants mentioned in tables 1a to 1d carry as a foreign gene at least one gene selected from the group of the foreign genes of the present invention, as defined above. In one embodiment of the present invention the at least one gene is selected from the group consisting of a gene encoding an antibody or its antigen-binding part directed to the protein PD-L1, such as Atezolizumab, an antigen-binding part thereof or a variant of Atezolizumab or its antigen-binding part, a gene encoding an antibody or its antigen-binding part directed to the growth factor protein VEGF-A, such as Bevacizumab, an antigen-binding part thereof or a variant of Bevacizumab or its antigen-binding part, and any combination of these genes or parts or variants thereof.

In describing a protein or peptide formulation, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal (left terminal to right terminal), the N-terminal being identified as a first residue. Amino acids are designated by their 3-letter abbreviation, 1-letter abbreviation, or full name, as follows. Ala:A:alanine; Asp:D:aspartic acid; Glu:E:glutamic acid; Phe:F:phenylalanine; Gly:G:glycine; His:H:histidine; Ile:I:isoleucine; Lys:K:lysine; Leu:L:leucine; Met:M:methionine; Asn:N:asparagine; Pro:P:proline; Gln:Q:glutamine; Arg:R:arginine; Ser:S:serine; Thr:T:threonine; Val:V:valine; Trp:W:tryptophan; Tyr:Y:tyrosine; Cys:C:cysteine.

In a preferred embodiment the NDV and the recombinant NDV resulting thereof is encoded by and/or comprises at least one of the nucleic acids according to SEQ ID No. 1 to 2 and SEQ ID No. 4-13 or parts thereof. According to another preferred the recombinant NDV strains of the present invention have a sequence identity of at least 75%, particularly of at least 90%, more particularly of at least 95% to any one of SEQ ID No. 1 to 2 and SEQ ID NO. 4 to 13. The sequences given by these SEQ ID numbers are as follows:
SEQ ID No. 1: Nucleic acid (cDNA) sequence of the Newcastle disease virus (NDV) strain MTH-68/H genome;
SEQ ID No. 2: Nucleic acid (cDNA) sequence of Newcastle disease virus strain Mut HN(F277L)/M(G165W) genome having a mutation in the HN gene and in the M gene (=NDV-NoThaBene-1 genome);
SEQ ID No. 3: Nucleic acid (cDNA) sequence of Newcastle disease virus strain Mut HN(F277L)/M(G165W)/F(F117S)/F(F190L)/L(V757I)/L(F1551S)/L(R1700L) genome having a mutation in the HN gene, a mutation in the M gene, two mutations in the F gene (F2) and three mutations in the L gene (L3) (=NDV-NoThaBene-2 genome);
SEQ ID No. 4: Nucleic acid (cDNA) sequence of Newcastle disease virus strain Mut HN(F277L)/M(G165W)-Atezolizumab genome created by reverse genetics as disclosed herein, wherein the nucleic acid comprises a mutation in the HN gene and in the M gene, and additionally encodes Atezolizumab;
SEQ ID No. 5: Nucleic acid (cDNA) sequence of Newcastle disease virus strain Mut HN(F277L)/M(G165W)-Bevacizumab genome created by reverse genetics as disclosed herein, wherein the nucleic acid comprises a mutation in the HN gene and in the M gene, and additionally encodes Bevacizumab;
SEQ ID No. 6: Nucleic acid (cDNA) sequence of Newcastle disease virus strain Mut HN(F277L)/M(G165W)-Lirilumab genome created by reverse genetics as disclosed herein, wherein the nucleic acid comprises a mutation in the HN gene and in the M gene, and additionally encodes Lirilumab;
SEQ ID No. 7: Nucleic acid (cDNA) sequence of Newcastle disease virus strain Mut HN(F277L)/M(G165W)-Relatlimab genome created by reverse genetics as disclosed herein, wherein the nucleic acid comprises a mutation in the HN gene and in the M gene, and additionally encodes Relatlimab;
SEQ ID No. 8: Nucleic acid (cDNA) sequence of Newcastle disease virus strain Mut HN(F277L)/M(G165W)-Monalizumab genome created by reverse genetics as disclosed herein, wherein the nucleic acid comprises a mutation in the HN gene and in the M gene, and additionally encodes Monalizumab;
SEQ ID No. 9: Nucleic acid (cDNA) sequence of Newcastle disease virus strain Mut HN(F277L)/M(G165W)-TRX518 genome created by reverse genetics as disclosed herein, wherein the nucleic acid comprises a mutation in the HN gene and in the M gene, and additionally encodes TRX518;
SEQ ID No. 10: Nucleic acid (cDNA) sequence of Newcastle disease virus strain Mut HN(F277L)/M(G165W)-BMS 986178 genome created by reverse genetics as disclosed herein, wherein the nucleic acid comprises a mutation in the HN gene and in the M gene, and additionally encodes BMS 986178;
SEQ ID No. 11: Nucleic acid (cDNA) sequence of Newcastle disease virus strain Mut HN(F277L)/M(G165W)-CD80 genome created by reverse genetics as disclosed herein, wherein the nucleic acid comprises a mutation in the HN gene and in the M gene, and additionally encodes CD80;
SEQ ID No. 12: Nucleic acid (cDNA) sequence of Newcastle disease virus strain Mut HN(F277L)/M(G165W)-(ns)hIL-12 genome created by reverse genetics as disclosed herein, wherein the nucleic acid comprises a mutation in the HN gene and in the M gene, and additionally encodes (ns)hIL-12;
SEQ ID No. 13: Nucleic acid (cDNA) sequence of Newcastle disease virus strain Mut HN(F277L)/M(G165W)-eGFP genome created by reverse genetics as disclosed herein, wherein the nucleic acid comprises a mutation in the HN gene and in the M gene, and additionally encodes eGFP;
SEQ ID No. 14: Nucleic acid (cDNA) sequence of Newcastle disease virus strain Mut HN(F277L)/M(G165W)/F(F117S)/F(F190L)/L(V757I)/L(F1551S)/L(R1700L)-Atezolizumab genome created by reverse genetics as disclosed herein, wherein the nucleic acid comprises a mutation in the HN gene, a mutation in the M gene, two mutations in the F gene (F2) and three mutations in the L gene (L3), and additionally encodes Atezolizumab;
SEQ ID No. 15: Nucleic acid (cDNA) sequence of Newcastle disease virus strain Mut HN(F277L)/M(G165W)/F(F117S)/F(F190L)/L(V757I)/L(F1551S)/L(R1700L)-Bevacizumab genome created by reverse genetics as disclosed herein, wherein the nucleic acid comprises a mutation in the HN gene, a mutation in the M gene, two mutations in the F gene (F2) and three mutations in the L gene (L3), and additionally encodes Bevacizumab;
SEQ ID No. 16: Nucleic acid (cDNA) sequence of Newcastle disease virus strain Mut HN(F277L)/M(G165W)/F(F117S)/F(F190L)/L(V757I)/L(F1551S)/L(R1700L)-Lirilumab genome created by reverse genetics as disclosed herein, wherein the nucleic acid comprises a mutation in the HN gene, a mutation in the M gene, two mutations in the F gene (F2) and three mutations in the L gene (L3), and additionally encodes Lirilumab;
SEQ ID No. 17: Nucleic acid (cDNA) sequence of Newcastle disease virus strain Mut HN(F277L)/M(G165W)/F(F117S)/F(F190L)/L(V757I)/L(F1551S)/L(R1700L)-Relatlimab genome created by reverse genetics as disclosed herein, wherein the nucleic acid comprises a mutation in the HN gene, a mutation in the M gene, two mutations in the F gene (F2) and three mutations in the L gene (L3), and additionally encodes Relatlimab;
SEQ ID No. 18: Nucleic acid (cDNA) sequence of Newcastle disease virus strain Mut HN(F277L)/M(G165W)/F(F117S)/F(F190L)/L(V757I)/L(F1551S)/L(R1700L)-Monalizumab genome created by reverse genetics as disclosed herein, wherein the nucleic acid comprises a mutation in the HN gene, a mutation in the M gene, two mutations in the F gene (F2) and three mutations in the L gene (L3), and additionally encodes Monalizumab;
SEQ ID No. 19: Nucleic acid (cDNA) sequence of Newcastle disease virus strain Mut HN(F277L)/M(G165W)/F(F117S)/F(F190L)/L(V757I)/L(F1551S)/L(R1700L)-TRX518 genome created by reverse genetics as disclosed herein, wherein the nucleic acid comprises a mutation in the HN gene, a mutation in the M gene, two mutations in the F gene (F2) and three mutations in the L gene (L3), and additionally encodes TRX518;
SEQ ID No. 20: Nucleic acid (cDNA) sequence of Newcastle disease virus strain Mut HN(F277L)/M(G165W)/F(F117S)/F(F190L)/L(V757I)/L(F1551S)/L(R1700L)-BMS 986178 genome created by reverse genetics as disclosed herein, wherein the nucleic acid comprises a mutation in the HN gene, a mutation in the M gene, two mutations in the F gene (F2) and three mutations in the L gene (L3), and additionally encodes BMS 986178;
SEQ ID No. 21: Nucleic acid (cDNA) sequence of Newcastle disease virus strain Mut HN(F277L)/M(G165W)/F(F117S)/F(F190L)/L(V757I)/L(F1551S)/L(R1700L)-CD80 genome created by reverse genetics as disclosed herein, wherein the nucleic acid comprises a mutation in the HN gene, a mutation in the M gene, two mutations in the F gene (F2) and three mutations in the L gene (L3), and additionally encodes CD80;
SEQ ID No. 22: Nucleic acid (cDNA) sequence of Newcastle disease virus strain Mut HN(F277L)/M(G165W)/F(F117S)/F(F190L)/L(V757I)/L(F1551S)/L(R1700L)-(ns)hIL-12 genome created by reverse genetics as disclosed herein, wherein the nucleic acid comprises a mutation in the HN gene, a mutation in the M gene, two mutations in the F gene (F2) and three mutations in the L gene (L3), and additionally encodes (ns)hIL-12;
SEQ ID No. 23: Nucleic acid (cDNA) sequence of Newcastle disease virus strain Mut HN(F277L)/M(G165W)/F(F117S)/F(F190L)/L(V757I)/L(F1551S)/L(R1700L)-eGFP genome created by reverse genetics as disclosed herein, wherein the nucleic acid comprises a mutation in the HN gene, a mutation in the M gene, two mutations in the F gene (F2) and three mutations in the L gene (L3), and additionally encodes eGFP.

A person skilled in the art is well aware of the fact that the nucleobase thymine in the nucleic acid sequence of DNA is replaced by the nucleobase uracil in the nucleic acid sequence of RNA.

That is in a preferred embodiment of the present invention the viral HN gene product has an amino acid sequence as identified in SEQ ID No. 24, and/or the viral M gene product preferably has an amino acid sequence as identified in SEQ ID No. 25.

Optionally, a NDV strain may further comprise beside the viral HN gene product having an amino acid sequence as identified in SEQ ID No. 24, and/or the viral M gene product preferably having an amino acid sequence as identified in SEQ ID No. 25, a viral F gene product having an amino acid sequence as identified in SEQ ID No. 26 and/or a viral L gene product having an amino acid sequence as identified in SEQ ID No. 27. Also encompassed by the present invention are NDV strains comprising variants of the gene products according to SEQ ID No. 24 to 25.

In another aspect the present invention also provides a recombinant nucleic acid comprising the nucleic acid sequence of the recombinant NDV strains of the present invention.

In describing nucleic acid formulation, structure and function herein, reference is made to any of a group of polymeric nucleotides such as DNA or RNA. Nucleic acid is composed of either one or two chains of repeating units called nucleotides, which nucleotides consist of a nitrogen base (a purine or pyrimidine) attached to a sugar phosphate. In the present specification, nucleotide residues are identified by using the following abbreviations. Adenine residue: A; guanine residue: G; thymine residue: T; cytosine residue: C; uracil residue: U. Also, unless explicitly otherwise indicated, the nucleotide sequences of nucleic acid are identified from 5′-terminal to 3′-terminal (left to right terminal), the 5′-terminal being identified as a first residue.

A nucleic acid according to the present invention comprises a transgenic construct. According to one embodiment the transgenic construct encodes an antibody directed to the protein PD-L1 or an antigen-binding part that is directed to the protein PD-L1. According to the present invention the encoded antibody is in a particularly preferred embodiment Atezolizumab, an antigen-binding part of Atezolizumab or a variant of Atezolizumab or its antigen-binding part. According to another embodiment the transgenic construct encodes an antibody directed to the growth factor protein VEGF-A or an antigen-binding part that is directed to the growth factor protein VEGF-A. According to the present invention the encoded antibody is in a particularly preferred embodiment Bevacizumab, an antigen-binding part of Bevacizumab or a variant of Bevacizumab or its antigen-binding part. According to another embodiment the transgenic construct encodes an antibody directed to the killer-cell immunoglobulin-like receptors (KIRs), in particular KIR2DL1/2L3, or an antigen-binding part that is directed to the killer-cell immunoglobulin-like receptors (KIRs), in particular KIR2DL1/2L3. According to the present invention the encoded antibody is in a particularly preferred embodiment Lirilumab, an antigen-binding part of Lirilumab or a variant of Lirilumab or its antigen-binding part. According to another embodiment the transgenic construct encodes an antibody directed to and inhibiting LAG-3, or an antigen-binding part that is directed to and inhibiting LAG-3. According to the present invention the encoded antibody is in a particularly preferred embodiment Relatlimab, an antigen-binding part of Relatlimab or a variant of Relatlimab or its antigen-binding part. According to another embodiment the transgenic construct encodes an antibody directed to NKG2A, or an antigen-binding part that is directed to NKG2A. According to the present invention the encoded antibody is in a particularly preferred embodiment Monalizumab, an antigen-binding part of Monalizumab or a variant of Monalizumab or its antigen-binding part. According to another embodiment the transgenic construct encodes an antibody directed to the glucocorticoid-induced TNF-superfamily receptor (GITR), or an antigen-binding part that is directed to the glucocorticoid-induced TNF-superfamily receptor (GITR). According to the present invention the encoded antibody is in a particularly preferred embodiment TRX518, an antigen-binding part of TRX518 or a variant of TRX518 or its antigen-binding part. According to another embodiment the transgenic construct encodes an antibody directed to and activating the protein OX40, or an antigen-binding part that is directed to and activating the protein OX40. According to the present invention the encoded antibody is in a particularly preferred embodiment BMS 986178, an antigen-binding part of BMS 986178 or a variant of BMS 986178 or its antigen-binding part. According to another embodiment the transgenic construct encodes a membrane protein which is the receptor for the proteins CD28 and CTLA-4, and which membrane protein is involved in the costimulatory signal essential for T-lymphocyte activation, or part thereof which is the receptor for the proteins CD28 and CTLA-4, and which membrane protein is involved in the costimulatory signal essential for T-lymphocyte activation. According to the present invention the encoded protein is in a particularly preferred embodiment CD80, a part of CD80 or a variant of CD80 or its part. According to another embodiment the transgenic construct encodes a human interleukin 12 (hIL-12), or part thereof. According to the present invention the encoded protein is in a particularly preferred embodiment (ns)hIL-12, a part of (ns)hIL-12 or a variant of (ns)hIL-12 or its part. According to another embodiment the transgenic construct encodes a green fluorescent protein or part thereof which can be used for the localization and monitoring of cells, in particular cancer cells. According to the present invention the encoded protein is in a particularly preferred embodiment eGFP, a part of eGFP or a variant of eGFP or its part.

According to a further embodiment of the present invention, the transgenic construct comprised in the nucleic acid may be a combination of at least two of the foreign genes or parts or variants thereof as mentioned supra.

In addition to the transgenic construct a nucleic acid according to the present invention comprises a mutation in the HN gene, said mutation allowing replication of the NDV in a cancer cell to a higher level than replication of an otherwise identical NDV not having said the mutation in the HN gene.

In a preferred embodiment of the present invention the sequence encoding the recombinant NDV comprises a mutated HN gene encoding hemagglutinin-neuramidase with an amino acid substitution at position 277 to an amino acid with a hydrophobic side chain other than phenylalanine. Still more preferred the mutated HN gene encodes for a hemagglutinin-neuramidase referred to as HN^{F277L}. That is the amino acid phenylalanine (F) at position 277 of the hemagglutinin-neuramidase is substituted to leucine (L).

In a particular preferred embodiment the nucleic acid is derived from the NDV strain MTH-68/H.

In another embodiment of the present invention the sequence encoding the recombinant NDV may also comprises a mutation in the M gene. Preferably the mutated M gene encodes a matrix protein with an amino acid substitution at position 165 to an amino acid with an aromatic side chain. Most preferred the mutated M gene encodes M^{G165W}. That is the amino acid glycine (G) at position 165 of the M gene product is substituted to the amino acid tryptophane (W) at the respective position. In one embodiment of the present invention the mutation in the M gene is comprised in combination with the mutation in the HN gene as specified supra.

In a particular preferred embodiment the nucleic acid is derived from the NDV strain MTH-68/H and comprises a mutation in the HN gene resulting in an amino acid with a hydrophobic side chain other than phenylalanine at amino acid position 277, and comprising a mutation in the M gene resulting in a matrix protein with an amino acid substitution at position 165 to an amino acid with an aromatic side chain. Still more preferred the mutated HN gene encodes for a hemagglutinin-neuramidase in which the amino acid phenylalanine (F) at position 277 of the hemagglutinin-neuramidase is substituted to leucine (L), and the mutated M gene encodes for a matrix protein in which the amino acid glycine (G) at position 165 of the M protein is substituted to the amino acid tryptophane (W).

Optionally, the sequence encoding the recombinant NDV comprises, in addition to the above described mutation in the HN gene, particularly the mutation resulting in an amino acid exchange or substitution at position 277 of the HN gene product as specified supra, optionally in combination with the above described mutation on the M gene, particularly the mutation resulting in an amino acid exchange at position 165 of the M gene product as specified supra, the nucleic acid sequence encoding the recombinant NDV also comprises at least one mutation in the F gene. Preferably the mutated F gene encodes a fusion protein with an amino acid substitution at position 117 and/or position 190. With regard to amino acid position 117 the amino acid is substituted to an amino acid with a hydroxylated side chain. Most preferred the mutated F gene encodes F^{F117s}. That is the amino acid phenylalanine (F) at position 117 of the F gene product is substituted to the amino acid serine (S) at the respective position. With regard to the amino acid substitution at position 190 the amino acid is changed to an amino acid with an aliphatic side chain. Most preferred the mutated F gene encodes F^{F190L}. That is the amino acid phenylalanine (F) at position 190 of the F gene product is substituted to the amino acid leucine (L) at the respective position. The combination of both mutations at amino acid position 117 (F^{F117S}) and 190 (F^{F190L}) is particularly preferred. Optionally the at least one mutation, preferably both mutations, in the F gene may further be comprised in combination with the mutation in the HN gene as specified supra and optionally, also in combination with the mutation in the M gene as specified supra.

In a particular preferred embodiment the nucleic acid is derived from the NDV strain MTH-68/H and comprises a mutation in the HN gene resulting in an amino acid with a hydrophobic side chain other than phenylalanine at amino acid position 277, a mutation in the M gene resulting in a matrix protein with an amino acid substitution at position 165 to an amino acid with an aromatic side chain, and optionally the NDV strain may further comprise at least one mutation in the F gene resulting in a fusion protein with an amino acid substitution at position 117 to an amino acid with a hydroxylated side chain and/or optionally an amino acid substitution at position 190 to an amino acid with an aliphatic side chain. Still more preferred the mutated HN gene encodes for a hemagglutinin-neuramidase in which the amino acid phenylalanine (F) at position 277 of the hemagglutinin-neuramidase is substituted to leucine (L), the optionally mutated M gene encodes for a matrix protein in which the amino acid glycine (G) at position 165 is substituted to the amino acid tryptophane (W), and the mutated F gene encodes for a fusion protein in which the amino acid phenylalanine (F) at position 117 is substituted to the amino acid serine (S) and/or the amino acid phenylalanine (F) at position 190 is substituted to the amino acid leucine (L). The combination of both mutations at amino acid position 117 (F^{F117S}) and 190 (F^{F190}L) of the F gene is particularly preferred.

Optionally, the sequence encoding the recombinant NDV may further comprise, in addition to the above described mutation in the HN gene, particularly the mutation resulting in an amino acid substitution at position 277 of the HN gene product as specified supra, and in addition to the above described at least one mutation in the F gene, particularly the mutation resulting in an amino acid substitution at position 117 (FF1175) and/or at position 190 (F^{F190L}) of the F gene product as specified supra, optionally in combination with the above described mutation on the M gene, particularly the mutation resulting in an amino acid exchange at position 165 of the M gene product as specified supra, also may comprise at least one mutation in the L gene. Preferably the mutated L gene encodes for a L protein with at least one amino acid substitution at amino acid position 757, 1551 and/or 1700. At position 757 the amino acid may be changed to an amino acid with an aliphatic side chain other than valine (V). Most preferred the mutated L gene encodes L^{V757I}. That is the amino acid valine (V) at position 757 of the L gene product is substituted to the amino acid isoleucine (I) at the respective position. At position 1551 the amino acid may be changed to an amino acid with a hydroxylated side chain. Most preferred the mutated L gene encodes L^{F1551S}. That is the amino acid phenylalanine (F) at position 1551 of the L gene product is substituted to the amino acid serine (S) at the respective position. At position 1700 the amino acid may be changed to an amino acid with an aliphatic side chain. Most preferred the mutated L gene encodes L^{R1700L}. That is the amino acid arginine (R) at position 1700 of the L gene product is substituted to the amino acid leucine (L) at the respective position. The mutations at amino acid positions 757, 1551 and 1700 of the L gene may be present alone or in each possible combination, i.e. position 757 and/or position 1551 and/or position 1700. The combination of all three mutations in the L gene (L3) is particularly preferred. Optionally, the at least one mutation in the L gene, preferably all three mutation at the respective amino acid positions 757, 1551 and 1700, may be comprised in combination with the mutation in the HN gene as specified supra, the one or two mutations in the F gene as specified supra, and optionally, also in combination with the mutation in the M gene as specified supra.

In a particular preferred embodiment the nucleic acid is derived from the NDV strain MTH-68/H and comprises a mutation in the HN gene resulting in an amino acid with a hydrophobic side chain other than phenylalanine at position 277 of the respective amino acid sequence, a mutation in the M gene resulting in a matrix protein with an amino acid substitution at position 165 to an amino acid with an aromatic side chain, optionally, the NDV strain may further comprise at least one mutation of the F gene resulting in a fusion protein with an amino acid substitution at position 117 to an amino acid with a hydroxylated side chain and/or an amino acid substitution at position 190 to an amino acid with an aliphatic side chain, and at least one mutation in the L gene resulting in a L protein with an amino acid substitution at position 757 to an amino acid with an aliphatic side chain other than valine (V), and/or an amino acid substitution at position 1551 to an amino acid with a hydroxylated side chain, and/or an amino acid substitution at position 1700 to an amino acid with an aliphatic side chain. More preferred, the mutated L gene results in a L protein comprising the three amino acid substitutions at positions 757, 1551 and 1700. Still more preferred the mutated HN gene encodes for a hemagglutinin-neuramidase in which the amino acid phenylalanine (F) at position 277 of the hemagglutinin-neuramidase is substituted to leucine (L), the optionally mutated M gene encodes for a matrix protein in which the amino acid glycine (G) at position 165 is substituted to the amino acid tryptophane (W), the mutated F gene encodes for a fusion protein in which the amino acid phenylalanine (F) at position 117 is substituted to the amino acid serine (S) and/or the amino acid phenylalanine (F) at position 190 is substituted to the amino acid leucine (L), and the mutated L gene encodes for a L protein in which the amino acid valine (V) at position 757 is substituted to the amino acid isoleucine (I), and/or the amino acid phenylalanine (F) at position 1551 is substituted to the amino acid serine (S), and/or the amino acid arginine (R) at position 1700 is substituted to the amino acid leucine (L). Still more preferred the L protein comprises all three mutations at positions 757, 1551 and 1700.

Optional are those nucleic acids , which may comprise, in addition to the mutation in the HN gene, preferably in combination with one or more mutation in the M gene, the F gene and the L gene, as specified supra, a mutation in the F gene resulting in an amino acid substitution at position 289 of the amino acid sequence, and/or at least one mutation in the L gene resulting in an amino acid substitution at position 1717 and/or at position 1910 of the L protein, and/or a mutation in the P gene as specified supra.

In one embodiment of the present invention the nucleic acid consists of or comprises at least one of the nucleic acids according to SEQ ID No. 2 and 4 to 13 or parts thereof. In a more preferred embodiment the nucleic acid of the present invention has a sequence identity of at least 75%, particularly of at least 90%, more particularly of at least 95% to any of the sequences according to SEQ ID No. 2 and 4 to 13.

In one embodiment of the present invention the nucleic acid is derived from a recombinant NDV as described supra.

The skilled artisan knows how to use the disclosed sequences to produce synthetic DNAs (e.g. vectors, in particular in rg-NDV vectors), and how to use the synthetic DNAs to reconstruct and express the viral genome. Thus, the skilled artisan is enabled to produce virus particles by using the information disclosed herein.

Recombinant viral genomes, which can be used to rescue virus particles, can for example be obtained by a method called ‘reverse genetics’. Therefore, in another aspect the present invention also provides a method (herein also called ‘reverse genetics’) for providing cloned full-length cDNA of the recombinant NDV strains according to the present invention, and, in a further embodiment, the invention also provides infectious virus particles obtained from said full-length cDNA of the recombinant NDV strains according to the present invention. These recombinant NDV strains have an unexpected beneficial replication capability in cell cultures, eggs or animals that are used to propagate the virus for pharmaceutical purposes, and/or have an improved viral safety profile, giving production advantages to such improved rgNDV strains. Furthermore, the higher replication rate is also particularly useful to obtain the desired oncolytic effects in vivo in that more virus particles will be produced for subsequent rounds of infection of cancer cells that were missed in the first round of infection. What is more, higher levels (yields) of the foreign gene or the foreign genes encoded by the transgenic construct will be achieved for providing the desired enhanced oncolytic effects in vivo. Thus, by modifying the HN protein within a transgene-expressing NDV higher yields of the transgenic construct protein can be obtained, which is very useful in anti-tumor immunotherapy. By optionally modifying the F protein and/or the L protein, as specified supra, also the viral safety can be improved.

The recombinant NDV strains according to the present invention can be obtained by a reverse genetic method for preparing an rgNDV encoding at least one foreign gene and having improved replication in a cancer cell, and preferably also improved viral safety, over a parent NDV.

The method according to the present invention comprises providing a nucleic acid construct encoding a HN gene with a mutation, particularly wherein the mutation in the HN gene leads to a change in the expression of the hemagglutinin-neuraminidase, wherein the amino acid, particularly phenylalanine (F), in position 277 is substituted, particularly wherein the amino acid in position 277 is substituted to an amino acid with a hydrophobic side chain, more particularly wherein the amino acid in position 277 is substituted to leucine (L) at position 277 of the HN gene product/HN protein.

The method further comprises a step of providing a nucleic acid encoding a rgNDV further comprising a transgenic construct encoding an antibody directed to the protein PD-L1 or an antigen-binding part that is directed to the protein PD-L1, preferably Atezolizumab, an antigen-binding part of Atezolizumab or a variant of Atezolizumab or its antigen-binding part, an antibody directed to the growth factor protein VEGF-A or an antigen-binding part that is directed to the growth factor protein VEGF-A, preferably Bevacizumab, an antigen-binding part of Bevacizumab or a variant of Bevacizumab or its antigen-binding part, an antibody directed to the killer-cell immunoglobulin-like receptors (KIRs), in particular KIR2DL1/2L3, or an antigen-binding part that is directed to the killer-cell immunoglobulin-like receptors (KIRs), in particular KIR2DL1/2L3, preferably Lirilumab, an antigen-binding part of Lirilumab or a variant of Lirilumab or its antigen-binding part, an antibody directed to and inhibiting LAG-3, or an antigen-binding part that is directed to and inhibiting LAG-3, preferably Relatlimab, an antigen-binding part of Relatlimab or a variant of Relatlimab or its antigen-binding part, an antibody directed to NKG2A, or an antigen-binding part that is directed to NKG2A, preferably Monalizumab, an antigen-binding part of Monalizumab or a variant of Monalizumab or its antigen-binding part, an antibody directed to the glucocorticoid-induced TNF-superfamily receptor (GITR), or an antigen-binding part that is directed to the glucocorticoid-induced TNF-superfamily receptor (GITR), preferably TRX518, an antigen-binding part of TRX518 or a variant of TRX518 or its antigen-binding part, an antibody directed to and activating the protein OX40, or an antigen-binding part that is directed to and activating the protein OX40, preferably BMS 986178, an antigen-binding part of BMS 986178 or a variant of BMS 986178 or its antigen-binding part, a membrane protein which is the receptor for the proteins CD28 and CTLA-4, and which membrane protein is involved in the costimulatory signal essential for T-lymphocyte activation, or part thereof which is the receptor for the proteins CD28 and CTLA-4, and which membrane protein is involved in the costimulatory signal essential for T-lymphocyte activation, preferably CD80, a part of CD80 or a variant of CD80 or its part, a human interleukin 12 (hIL-12), or part thereof, preferably (ns)hIL-12, a part of (ns)hIL-12 or a variant of (ns)hIL-12 or its part, and/or a green fluorescent protein, or part thereof, preferably eGFP, a part of eGFP or a variant of eGFP or its part.

In the next step of the claimed method the nucleic acid construct with said mutation in the HN gene (optionally with further mutations, in particular those as specified herein) is incorporated in the nucleic acid encoding a rgNDV comprising the transgenic construct in order to obtain a full length cDNA of the NDV, which additionally comprises a transgenic construct as specified supra. In a preferred embodiment this method step results in a nucleic acid as disclosed supra.

The nucleic acid encoding a rgNDV further comprising a transgenic construct as specified supra can be obtained by generating sub-genomic cDNA fragments and assembling the full length cDNA of the recombinant NDV from these fragments.

Following these method steps the thus obtained nucleic acid encoding a recombinant and mutated rgNDV according to the present invention is used to produce infectious rgNDV particles, which replication characteristics and expression rates of the encoded foreign protein(s) in cancer cells are compared with the replication characteristics and expressions rates of the parent NDV used for designing the recombinant and mutated NDV strain.

Finally, said rgNDV is selected for further use, when it shows an improved replication characteristic over the parent NDV and/or an improved viral safety, as well as a sufficient expression of the gene product or gene products of the at least one foreign genes.

If it is intended to introduce further to the mutation in the HN gene, at least one further mutation in a viral gene, preferably the M gene, optionally the F gene, optionally the L gene and/or the P gene, the selected recombinant and mutated rgNDV comprising a transgenic construct as specified supra and a mutated HN gene is used for incorporating a nucleic acid construct encoding a mutated M gene, F gene, L gene or P gene, and the method for introducing the mutation in the HN gene is repeated respectively. Alternatively there is already provided a nucleic acid encoding a rgNDV with the transgenic construct and carrying a mutation in the M gene, F gene, L gene or P gene, particularly wherein said mutation is capable of improving oncolytic potential of said rgNDV.

According to a preferred embodiment of the present invention the mutation in the M gene comprises or is a mutation which encodes a matrix protein with an amino acid substitution at position 165 to an amino acid with an aromatic side chain. Still more preferred is a substitution of the amino acid glycine (G) at position 165 of the M gene product to the amino acid tryptophane (W).

Optionally, the at least one mutation in the F gene comprises or is a mutation which encodes a fusion protein/F protein with an amino acid substitution at position 117 to an amino acid with a hydroxylated side chain and/or with an amino acid substitution at position 190 to an amino acid with an aliphatic side chain. Preferred is a substitution of the amino acid phenylalanine (F) at position 117 of the F gene product to the amino acid serine (S) and/or a substitution of the amino acid phenylalanine (F) at position 190 of the F gene product to the amino acid leucine (L). Optionally, the F gene may also comprise a mutation which encodes a fusion protein with an amino acid substitution at position 289 to an amino acid with an aliphatic side chain other than leucine, preferably a substitution of the amino acid leucine (L) at position 289 of the F gene product to the amino acid alanine (A).

Optionally, the at least one mutation in the L gene comprises or is a mutation which encodes a L protein with an amino acid substitution at position 757 to an amino acid with an aliphatic side chain other than valine, and/or with an amino acid substitution at position 1551 to an amino acid with a hydroxylated side chain, and/or with an amino acid substitution at position 1700 to an amino acid with an aliphatic side chain. Still more preferred is a substitution of the amino acid valine (V) at position 757 of the L gene product to the amino acid isoleucine (I) and/or a substitution of the amino acid phenylalanine (F) at position 1551 of the L gene product to the amino acid serine (S), and/or a substitution of the amino acid arginine (R) at position 1700 of the L gene product to the amino acid leucine (L). Optionally, the L gene may also comprise a mutation which encodes a L protein with an amino acid substitution at position 1717 to an amino acid with an aromatic side chain other than tyrosine, preferably a substitution of the amino acid tyrosine (Y) at position 1717 of the L gene product to the amino acid histidine (H), and/or a mutation which encodes a L protein with an amino acid substitution at position 1910 to an amino acid with a basic side chain, preferably a substitution of the amino acid glutamic acid (E) at position 1910 of the L gene product to the amino acid lysine (K).

According to another preferred embodiment of the present invention the mutation is in the RNA-editing sequence of the P gene. More preferably, the mutation abolishes and/or decreases the expression of the V protein.

It is also within the scope of the present invention that the viral genome comprises a combination of two or more mutations, which are selected from a mutation in the HN gene, a mutation in the M gene, optionally at least one mutation in the F gene, optionally at least one mutation in the L gene, and a mutation in the P gene. For the details of the respective mutations, reference to the detailed disclosure of each of these mutations given supra and in tables 1 is made.

The invention also provides an isolated, recombinant nucleic acid encoding a rgNDV obtainable by a method for preparing an rgNDV having improved replication in a cancer cell in comparison to that of a parent NDV, as described supra.

In one preferred embodiment the isolated and recombinant nucleic acid encodes a rgNDV provided with a mutation in the HN gene resulting in a change of the amino acid phenylalanine (F) at amino acid position 277 of the hemagglutinin-neuraminidase into a leucine (L), said mutation (a F277L mutation) allowing replication of said rgNDV in a human cancer cell to a higher level than replication of an otherwise identical rgNDV having the amino acid phenylalanine (F) at position 277 in the HN gene product in combination with a transgenic construct comprising any one of the foreign gene of the present invention, as defined supra. For example the said isolated and recombinant nucleic acid encoding an antibody or an antigen-binding part thereof directed to the protein PD-L1, preferably Atezolizumab, an antigen-binding part thereof or a variant of Atezolizumab or its antigen-binding part. The rgNDV may further comprise at least one or more mutation(s) selected from a mutation in the M gene, preferably G165W, optionally a mutation at amino acid position 117, 190 and/or 289 in the F gene, preferably F117S, F190L, and/or L289A, optionally a mutation at amino acid position 757, 1551, 1700, 1717 and/or 1910 in the L gene, preferably V757I, F1551S, R1700L, Y1717H and/or E1910K, and a mutation in the P gene, preferably the mutation in the P gene abolishes and/or decreases the expression of the V protein.

In another much preferred embodiment the isolated and recombinant nucleic acid encodes a rgNDV provided with a mutation in the HN gene resulting in a change of the amino acid phenylalanine (F) at amino acid position 277 of the hemagglutinin-neuraminidase into a leucine (L), said mutation (a F277L mutation) allowing replication of said rgNDV in a human cancer cell to a higher level than replication of an otherwise identical rgNDV having the amino acid phenylalanine (F) at position 277 in the HN gene in combination with a transgenic construct encoding an antibody or an antigen-binding part thereof directed to the growth factor protein VEGF-A, preferably Bevacizumab, an antigen-binding part thereof or a variant of Bevacizumab or its antigen-binding part. The rgNDV may further comprise at least one or more mutation(s) selected from a mutation in the M gene, preferably G165W, optionally a mutation at amino acid position 117, 190 and/or 289 in the F gene, preferably F117S, F190L, and/or L289A, optionally a mutation at amino acid position 757, 1551, 1700, 1717 and/or 1910 in the L gene, preferably V7571, F1551S, R1700L, Y1717H and/or E1910K, and a mutation in the P gene, preferably the mutation in the P gene abolishes and/or decreases the expression of the V protein.

According to a particularly preferred embodiment the nucleic acid used to engineer a recombinant NDV genome is derived from the NDV strain MTH-68/H. Thus the present invention provides in one embodiment nucleic acids, preferably obtainable by the above specified method, which are rgNDV-Mut HN(F277L)-Atezolizumab, rgNDV-Mut HN(F277L)-Bevacizumab, rgNDV-Mut HN(F277L)/M(G165W)-Atezolizumab (=rgNDV-NoThaBene-1-Atezolizumab), rgNDV-Mut HN(F277L)/M(G165W)-Bevacizumab (=rgNDV-NoThaBene-1-Bevacizumab). Optionally the NDV strains may further provide nucleic acids, preferably obtainable by the above specified method, which are rgNDV-Mut HN(F277L)/M(G165W)/F(F117S)/F(F190L)/L(V7571)/L(F1551S)/L(R1700L)-Atezolizumab (=rgNDV-NoThaBene-2-Atezolizumab), rgNDV-Mut HN(F277L)/M(G165W)/F(F117S)/F(F190L)/L(V757I)/L(F1551S)/L(R1700L)-Bevacizumab (=rgNDV-NoThaBene-2-Bevacizumab).

Other embodiments of the nucleic acid as provided by the present invention are:
rgNDV-Mut HN(F277L)-Lirilumab,
rgNDV-Mut HN(F277L)/M(G165W)-Lirilumab,
optionally rgNDV-Mut HN(F277L)/M(G165W)/F(F117S)/F(F190L)/L(V7571)/L(F1551S)/L(R1700L)-Lirilumab,
rgNDV-Mut HN(F277L)-Relatlimab,
rgNDV-Mut HN(F277L)/M(G165W)-Relatlimab,
optionally rgNDV-Mut HN(F277L)/M(G165W)/F (F117 S)/F (F190L)/L(V7571)/L(F1551S)/L(R1700L)-Relatlimab,
rgNDV-Mut HN(F277L)-Monalizumab,
rgNDV-Mut HN(F277L)/M(G165W)-Monalizumab,
optionally rgNDV-Mut HN(F277L)/M(G165W)/F (F117 S)/F (F190L)/L(V7571)/L(F1551S)/L(R1700L)-Monalizumab,
rgNDV-Mut HN(F277L)-TRX518,
rgNDV-Mut HN(F277L)/M(G165W)-TRX518,
optionally rgNDV-Mut HN(F277L)/M(G165W)/F(F117S)/F(F190L)/L(V7571)/L(F1551S)/L(R1700L)-TRX518,
rgNDV-Mut HN(F277L)-BMS 986178,
rgNDV-Mut HN(F277L)/M(G165W)-BMS 986178,
optionally rgNDV-Mut HN(F277L)/M(G165W)/F (F117 S)/F (F190L)/L(V7571)/L(F1551S)/L(R1700L)-BMS 986178,
rgNDV-Mut HN(F277L)-CD80,
rgNDV-Mut HN(F277L)/M(G165W)-CD 80,
optionally rgNDV-Mut HN(F277L)/M(G165W)/F (F117 S)/F (F190L)/L(V7571)/L(F1551S)/L(R1700L)-CD80,
rgNDV-Mut HN(F277L)-(ns)hIL-12,
rgNDV-Mut HN(F277L)/M(G165W)-(ns)hIL-12,
optionally rgNDV-Mut HN(F277L)/M(G165W)/F (F117 S)/F (F190L)/L(V7571)/L(F1551S)/L(R1700L)-(ns)hIL-12,
rgNDV-Mut HN(F277L)-eGFP,
rgNDV-Mut HN(F277L)/M(G165W)-eGFP,
optionally rgNDV-Mut HN(F277L)/M(G165W)/F (F117 S)/F (F190L)/L(V7571)/L(F1551S)/L(R1700L)-eGFP.

A NDV or rgNDV according to the present invention can be provided in a suitable cell or cell line, for example a HeLa cell line or in cancer cells, or an embryonated egg that is susceptible to a NDV infection.

In these infected cells the virus is grown to sufficient quantities, and preferably under sufficient conditions such that the virus is free from exogenous contamination, and the progeny virus particles are collected by suitable methods well known to those skilled in the art.

In a further aspect the present invention is concerned with the medical use of the recombinant NDV strains (the virus particles) of the present invention. In this regard the invention provides an oncolytic NDV for use in oncological treatment (this term is used herein also replaceable with “for use as a medicament, especially in a method of treatment of cancer”; or the use of said oncolytic NDV in the preparation of a pharmaceutical formulation for use in said method of treatment) in humans (or more generically animals, such as mammalian animals).

In this regard the present invention is first concerned with a recombinant NDV according to the present invention for use in medicine. Secondly, the present invention is more particularly concerned with a recombinant NDV according to the present invention for use in a method of treating cancer in a subject considered in need thereof.

In particular one or more of the recombinant and mutated NDV strains of the present invention are used in a method for treating one or more indications selected from the group consisting of brain tumors, like glioblastoma, bone tumors, like osteosarcoma and/or Ewing's sarcoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose, throat tumors, tongue tumors, and skin tumors, like melanoma.

In a preferred embodiment of the present invention the subject to be treated is a mammal, particularly a mammalian animal or a human subject. Still more preferred the one or more oncolytic NDV strains of the present invention may be used for the treatment of adults and/or children, preferably human adults and/or human children.

In one embodiment only one recombinant and mutated NDV or rgNDV strain according to the present invention is used in a method for treating cancer, in particular a cancer selected from the specific cancers as described and/or mentioned in the claims herein. Alternatively in another preferred embodiment of the present invention a combination of at least two of the recombinant and mutated NDV or rgNDV strains according to the present invention is used in a method for treating cancer, in particular a cancer selected from the specific cancers as described and/or mentioned in the claims herein. In particular it is preferred to use a combination of virus particles, wherein each virus particle encodes and expresses another foreign gene selected from the group of the foreign genes of the present invention, as defined above. For example, the foreign gene encoding an antibody directed to the protein PD-L1 or an antigen-binding part thereof directed to the protein PD-L1, preferably Atezolizumab, an antigen-binding part thereof or a variant of Atezolizumab or its antigen-binding part, or encoding an antibody directed to the growth factor protein VEGF-A or an antigen-binding part thereof directed to the growth factor protein VEGF-A, preferably Bevacizumab, an antigen-binding part thereof or a variant of Bevacizumab or its antigen-binding part. The use of such a combination of foreign genes results in an active combination of proteins beneficially improving the immunological response with the cancer.

Also within the scope of the present invention is the use of one or more NDV or rgNDV strains according to the present invention in combination with one or more other therapies suitable for the treatment of cancer. In a preferred embodiment the one or more NDV or rgNDV strains or a pharmaceutical formulation comprising the same is used in a method for treating cancer, especially a cancer selected from the specific cancers as described and/or mentioned in the claims herein.

The one or more NDV or rgNDV strains and one or more other therapies can be used concurrently or sequentially. In certain embodiments, the one or more NDV or rgNDV and the one or more other therapies are administered in the same (“fixed”) pharmaceutical formulation. In other embodiments, the one or more NDV or rgNDV strains and the one or more other therapies are administered in different formulations. The one or more NDV or rgNDV strains of the present invention and the one or more other therapies can be administered by the same or different routes of administration to the subject considered in need thereof. Another therapy within the meaning of the present invention also comprises an administration of other oncolytical virus strains, for example recombinant NDV or rgNDV strains encoding and expressing foreign genes other than those as disclosed within the present invention. Another foreign gene expressed by the NDV or rgNDV may be selected from the group consisting of a gene encoding an antibody directed to protein PD-1 (anti-PD-1), preferably a gene encoding Nivolumab, a gene encoding an antibody directed to the surface protein CTLA-4 (anti-CTLA-4), preferably a gene encoding Ipilimumab, a gene encoding a protein which improves the cellular immune response and the ability of T cells to enter tumor cells, preferably a gene encoding interleukin-12 (IL-12), a gene encoding a protein with the ability to modulate the virus replication cycle, preferably a gene encoding the non-structural protein NS1 of influenza A virus, a gene encoding a protein with the ability to selectively induce apoptosis in human tumor cells, but not in normal human cells, preferably a gene encoding Apoptin, a gene encoding a protein that reduces or inhibits IFN expression such as an IFN-beta receptor, preferably a gene encoding the viral protein B18R from vaccinia virus, any antigen-binding part of these antibodies, any parts of these other proteins, variants of these antibodies and these other proteins, and variants of these antigen-binding parts or parts. Particularly preferred NDV strains and rgNDV which may be used in combination are disclosed in the copending European patent application no. 18166400.4 and the copending European patent application no. 19154204.2. For details of these strains reference to these patent applications is made.

Particularly preferred is the combined use of recombinant NDV or rgNDV strains encoding and expressing as foreign genes a gene encoding an antibody directed to protein PD-1 or an antigen-binding part directed to protein PD-1 (anti-PD-1), preferably a gene encoding Nivolumab, an antigen-binding part of Nivolumab, a variant of Nivolumab or a variant of an antigen-binding part of Nivolumab, a recombinant NDV or rgNDV strain encoding and expressing as foreign genes a gene encoding an antibody directed to the surface protein CTLA-4 or an antigen-binding part directed to protein CTLA-4 (anti-CTLA-4), preferably a gene encoding Ipilimumab, an antigen-binding part of Ipilimumab, a variant of Ipilimumab or a variant of an antigen-binding part of Ipilimumab, and a recombinant NDV or rgNDV strain encoding and expressing as foreign genes a gene encoding an antibody directed to protein PD-L1 or an antigen-binding part directed to protein PD-L1 (anti-PD-L1), preferably a gene encoding Atezolizumab, an antigen-binding part of Atezolizumab, a variant of Atezolizumab or a variant of an antigen-binding part of Atezolizumab. Such a combination results in decreased side effects, as compared to directly administering the respective proteins to a patient in need thereof. For example the combination of the following NDV strains may be used in accordance with the present invention:
NDV-Mut HN(F277L)/M(G165W)-Nivolumab,
NDV-Mut HN(F277L)/M(G165W)-Ipilimumab, and
Optionally, NDV-Mut HN(F277L)/M(G165W)/F(F117S)/F(F190L)/L(V757I)/L(F1551S)/L(R1700L)-Atezolizumab

Or optionally,
NDV-Mut HN(F277L)/M(G165W)/F(F117S)/F(F190L)/L(V757I)/L(F1551S)/L(R1700L)-Nivolumab,
NDV-Mut HN(F277L)/M(G165W)/F(F117S)/F(F190L)/L(V757I)/L(F1551S)/L(R1700L)-Ipilimumab, and
NDV-Mut HN(F277L)/M(G165W)/F(F117S)/F(F190L)/L(V757I)/L(F1551S)/L(R1700L)-Atezolizumab

In a further aspect the present invention also provides a pharmaceutical formulation comprising a recombinant and mutated NDV strain, preferably an rgNDV, as described herein, without or preferably with at least one pharmaceutically acceptable carrier material.

For the indications mentioned herein, the appropriate dosage will, of course, vary depending upon, for example, the particular molecule of the invention to be employed, the mode of administration and the nature and severity of the condition being treated. In general, the dosage, in one embodiment of the invention, can preferably be in the range of 10⁷ to 10⁹ pfu per dose.

The recombinant and mutated NDV strains according to the present invention provide on the one hand an improved replication capacity of the NDV particles in cancer cells and/or an improved safety profile, preferably in humans, which is associated with increased cancer cell lysis and increased anti-cancer activity, because the recombinant and mutated NDV strains of the present invention are still selective for cancer cells. The low to sero activity towards normal cells is associated with an increased therapeutic window or safety margin, as is commonly determined for cancer therapeutics. Beside the virological activity the recombinant mutated NDV strains of the present invention are in addition beneficial for the treatment of cancer from an immunological point of view. This is because the foreign gene carried by NDV strains of the present invention beneficially influences the ability of the immune system of the subject to be treated to attack and destroy cancer cells. Advantageously the foreign gene is expressed inside the tumor to be treated, that is directly in the side of need.

The pharmaceutical formulations or pharmaceutical compositions of the present invention may be manufactured in any conventional manner, which are known to those skilled in the art. For example a pharmaceutical composition according to the present invention comprising a recombinant virus particle as described within this description can be provided in a dried, preferably in a lyophilized form and can be complemented with a suitable solvent, e.g. an aqueous carrier for injection at the time when used for administration. The dried form is very stable. Thus, in one embodiment the pharmaceutical composition is present as a dried form, still more preferred in a lyophilized form, and is reconstituted before administration by adding a suitable solvent. That is the present invention also relates to the above described dried form of the pharmaceutical composition for use as a medicament, preferably for use in a method of treating cancer. The process parameters for drying, preferably lyophilisation must be chosen such that the dried/lyophilized product contains live, replication competent viral particles, which can be grown to standardized titers, when needed. A person skilled in the art knows how to set these parameters.

For administration of a dried form, the dried form is directly dissolved in a suitable solvent or pharmaceutically acceptable carrier for injection and/or stabilization of the NDV or rg NDV and/or for improving its delivery to cancer cells. The solvent or carrier is preferably an aqueous carrier, for example sterile water for injection or sterile buffered physiological saline or another, preferably isotonic buffer system having a pH in the range of 7.2 to 7.6. The reconstitution can be performed ideally at or close to the intended time of administration in order to avoid any contaminations with microbes, etc. The skilled person is familiar with the handling of pharmaceutical compositions for reconstitution and reconstituted solutions.

In another embodiment, the present invention is concerned with pharmaceutical formulations comprising cancer cells infected with a transgene-expressing NDV or transgene-expressing rgNDV strain as described herein, and a pharmaceutically acceptable carrier. In specific embodiments, the cancer cells have been treated with gamma radiation prior to incorporation into the pharmaceutical formulation. In specific embodiments, the cancer cells have been treated with gamma radiation before infection with the NDV strain (e.g., rgNDV). In other specific embodiments, the cancer cells have been treated with gamma radiation after infection with the NDV (e.g., rgNDV). In another embodiment, presented herein are pharmaceutical formulations comprising a protein concentrate from lysed NDV-infected cancer cells (e.g., rgNDV infected cancer cells), and a pharmaceutically acceptable carrier.

The pharmaceutical formulation of the present invention can be prepared by a method comprising or consisting of the steps of:

propagating an NDV, preferably an rgNDV, according to the present invention in at least one cell or embryonated egg that is susceptible to a NDV infection;

collecting the progeny virus particles, wherein the virus is grown to sufficient quantities and under sufficient conditions that the virus is free from exogenous contamination, such that the progeny virus is suitable for formulation into a pharmaceutical formulation; and

preferably manufacturing a pharmaceutical formulation comprising said progeny virus particles in the absence of or after addition of at least one pharmaceutically acceptable carrier material.

Thus in a further aspect the present invention also provides a method for producing the above mentioned pharmaceutical formulation. It is also within the scope of the present invention that for manufacturing the said pharmaceutical formulation a nucleic acid according to the present invention is used, from which virus particles can be rescued which are then propagated in at least one cell or embryonated egg that is susceptible to a NDV infection.

The step of collecting progeny virus particles may in one embodiment also comprise a step of processing the collected virus-containing material to enrich virus particles and/or to eliminate host cell DNA.

In a further aspect the present invention also provides a cell or cell line or an embryonated egg comprising a NDV, preferably a rgNDV, according to the present invention.

In still a further aspect the present invention is also concerned with a method for treating cancer, especially a cancer selected from the specific cancers as described and/or mentioned in the claims herein, in a subject considered in need thereof. The method for treating cancer utilizes a transgene-expressing NDV or transgene-expressing rgNDV described herein or any combination thereof or a pharmaceutical formulation comprising such a NDV or rgNDV or any combination thereof, especially in a therapeutically effective amount. Within the meaning of the present patent application a therapeutically effective amount also includes an effective amount for preventing a cancer, in particular a cancer selected from the specific cancers as described and/or mentioned in the claims herein.

The method of treatment comprises or consists of administering to a subject in need thereof a NDV or an rgNDV according to the present invention or any combination thereof in a sufficient amount for infecting and destroying some or all of the cancer cells. In a specific embodiment the method for treating cancer comprises or consists of infecting a cancer cell in a subject with a NDV or rgNDV of the present invention or any combination thereof or with a pharmaceutical composition of the present invention. In another embodiment the one or more NDV or rgNDV or a pharmaceutical composition comprising the same is administered to a subject in need thereof by intravenous, intra-arterial, intratumoral, intramuscular, intradermal, subcutaneous, or any other medically relevant route of administration. According to another embodiment of the present invention the NDV or rgNDV according to the present invention or any combination thereof is administering to a subject in need thereof by administering cancer cells infected with a NDV or rgNDV according to the present invention, especially in a therapeutically effective amount, or pharmaceutical formulation thereof by intravenous, intra-arterial, intratumoral, intramuscular, intradermal, subcutaneous, or any other medically relevant route of administration. In specific embodiments, the cancer cells have been treated with gamma radiation prior to administration to the subject or incorporation into the pharmaceutical formulation. In still another embodiment, a method for treating cancer comprises or consists of administering to a subject in need thereof protein concentrates or plasma membrane fragments from cancer cells infected with a NDV or rgNDV or a pharmaceutical formulation according to the present invention.

In a particular preferred embodiment the method for treating a cancer, in particular a cancer selected from the specific cancers as described and/or mentioned in the claims herein, comprises administering a mixture of at least two recombinant and mutated NDV or rgNDV strains according to the present invention. In particular it is preferred to use a combination of virus particles, wherein each virus particle encodes and expresses another foreign gene selected from the group the foreign genes of the present invention, as defined above. For example, the encoded and expressed foreign genes are a gene encoding an antibody directed to the protein PD-L1 or an antigen-binding part directed to the protein PD-L1, preferably Atezolizumab, an antigen-binding part of Atezolizumab or a variant of Atezolizumab or its antigen-binding part, and a gene encoding an antibody directed to the growth factor protein VEGF-A or an antigen-binding part directed to the growth factor protein VEGF-A, preferably Bevacizumab, an antigen-binding part of Bevacizumab or a variant of Bevacizumab or its antigen-binding part. The administration of such a combination of foreign genes results in an active combination of proteins beneficially improving the immunological response with the cancer. The administration of this mixture of NDVs or rgNDVs according to the present invention can be achieved as specified supra.

Also within the scope of the present invention is a method of treating cancer, especially a cancer selected from the specific cancers as described and/or mentioned in the claims herein, which utilizes a NDV or rgNDV according to the present invention in combination with one or more other therapies. In a preferred embodiment the NDV or rgNDV or a pharmaceutical formulation comprising the same is administered to a subject in need thereof in a therapeutically effective amount. The NDV or rgNDV and one or more other therapies can be administered concurrently or sequentially to the subject (meaning they are jointly therapeutically active). In certain embodiments, the NDV or rgNDV and one or more other therapies are administered in the same (“fixed”) pharmaceutical formulation. In other embodiments, the NDV or rgNDV and one or more other therapies are administered in different formulations. The NDV or rgNDV of the present invention and one or more other therapies can be administered by the same or different routes of administration to the subject considered in need thereof. Another therapy within the meaning of the present invention also comprises an administration of other oncolytical virus strains, for example recombinant NDV or rgNDV strains encoding and expressing foreign genes other than those as disclosed within the present invention. Another foreign gene expressed by the NDV or rgNDV may be selected from the group consisting of a gene encoding an antibody directed to protein PD-1 (anti-PD-1), preferably a gene encoding Nivolumab, a gene encoding an antibody directed to the surface protein CTLA-4 (anti-CTLA-4), preferably a gene encoding Ipilimumab, a gene encoding a protein which improves the cellular immune response and the ability of T cells to enter tumor cells, preferably a gene encoding interleukin-12 (IL-12), a gene encoding a protein with the ability to modulate the virus replication cycle, preferably a gene encoding the non-structural protein NS1 of influenza A virus, a gene encoding a protein with the ability to selectively induce apoptosis in human tumor cells, but not in normal human cells, preferably a gene encoding Apoptin, a gene encoding a protein that reduces or inhibits IFN expression such as an IFN-beta receptor, preferably a gene encoding the viral protein B18R from vaccinia virus, any antigen-binding part of these antibodies, any parts of these other proteins, variants of these antibodies and these other proteins, and variants of these antigen-binding parts or parts. Particularly preferred NDV strains and rgNDV which may be used in combination are disclosed in the copending European patent application no. 18166400.4 and the copending European patent application no. 19154204.2. For details of these strains reference to these patent applications is made. Particularly preferred is the combined use of recombinant NDV or rgNDV strains encoding and expressing as foreign genes anti-PD-1, preferably Nivolumab, anti-CTLA-4, preferably Ipilimumab, anti-PD-L1, preferably Atezolizumab, or parts or variants thereof. For example the following combinations are in accordance with the present invention:
NDV-Mut HN(F277L)/M(G165W)-Nivolumab,
NDV-Mut HN(F277L)/M(G165W)-Ipilimumab, and
optionally NDV-Mut HN(F277L)/M(G165W)/F(F117S)/F(F190L)/L(V7571)/L(F1551S)/L(R1700L)-Atezolizumab

Or optionally,
NDV-Mut HN(F277L)/M(G165W)/F(F117S)/F(F190L)/L(V757I)/L(F1551S)/L(R1700L)-Nivolumab,
NDV-Mut HN(F277L)/M(G165W)/F(F117S)/F(F190L)/L(V7571)/L(F1551S)/L(R1700L)-Ipilimumab, and
NDV-Mut HN(F277L)/M(G165W)/F(F117S)/F(F190L)/L(V7571)/L(F1551S)/L(R1700L)-Atezolizumab

Another therapy may also comprise or may be radiotherapy for cancer.

### FIGURES

FIG. 1 is a schematic presentation of the NDV reverse genetics system. The upper part shows the composition of the full-length cDNA plasmid which contains the full-length NDV cDNA (encoding the nucleocapsid protein (NP), the phosphoprotein (P), the matrix protein (M), the fusion protein (F), the hemagglutinin-neuraminidase (HN), and the RNA-dependent RNA polymerase (L)) cloned behind the bacteriophage T7 RNA Polymerase promoter (T7P; yellow triangle) and followed by a ribozyme sequence (Rz) and T7 transcription termination signal (T7T).

A suitable host cell (shaded round-cornered box) is infected with a recombinant Fowlpox virus that expresses T7 DNA-dependent RNA polymerase (Fowlpox-T7) and subsequently co-transfected with the full-length cDNA plasmid and three helper plasmids containing the genes encoding the NDV NP, P and L proteins, respectively. Transcription of the full-length cDNA results in the generation of the NDV antigenome RNA which is encapsidated by NP protein then transcribed and replicated by the RNA-leading to the generation of infectious NDV.

FIG. 2 is a schematic presentation of the genome of the rgNDV-mutants according to the present invention. The figure shows the position of the respective foreign gene which is inserted as extra transcription unit into the rgNDV-mutant genome between the P and M genes.

FIG. 3 shows growth kinetics in HeLa cells. HeLa cells were infected at a multiplicity of infection (MOI) of 0.01 with the indicated viruses. At 0 h, 8 h, 24 h and 48 h after infection, the amount of infectious virus in the supernatant was determined by end-point titration on QM5 cells. MTH68: NDV strain MTH-68/H; MutHu: NDV-Mut HN(F277L)/M(G165W); rgMTH68: NDV strain MTH-68/H derived by reverse genetics from cloned full-length cDNA; rgMutHu: NDV strain NDV-Mut HN(F277L)/M(G165W) derived by reverse genetics from cloned full-length cDNA; rgMutHu(HNL277F): rgMutHu in which the amino acid mutation at position 277 in the HN gene was converted from L back to F; rgMutHu(MVV165G): rgMutHu in which the amino acid mutation at position 165 in the M gene was converted from W back to G.

FIG. 4 shows a calibration curve of a human IgG ELISA used for determining and quantifying the expression of Atezolizumab and Bevacizumab from a recombinant NDV strain according to the present invention.

### EXAMPLES

### Example 1

### Nucleotide Sequence Analysis of Mutant NDV-Mut HN(F277L)/M(G165W)

We identified a spontaneous mutant of an oncolytic NDV strain MTH-68/H (Csatary et al., 1999, Anticancer Res. 19:635-638.; further called MTH68). The replication capacity of the mutant strain (designated NDV-Mut HN(F277L)/M(G165W) in a variety of human neoplastic cell lines, as well as autologous primary tumors, is greatly enhanced as compared to the original MTH-68/H strain (also referred to as MTH68 strain). We analyzed its nucleotide sequence and found that, compared to MTH68, NDV-HN(F277L)/M(G165W) has two nucleotide mutations, one leading to an amino acid substitution in the M protein (G165W) and the other in the HN protein (F277L).

### Example 2

### A Reverse Genetics System that Allows Genetic Modification of NDV-Strains

2.1 Reverse Genetics

In order to be able to genetically modify the genome of an RNA virus such as NDV, a manipulatable genetic system must be developed that uses a copy of the full viral RNA (vRNA) genome in the form of DNA. This full-length cDNA is amenable to genetic modification by using recombinant DNA techniques. The authentic or modified cDNA can be converted back into vRNA in cells, which in the presence of the viral replication proteins results in the production of a new modified infectious virus. Such ‘reverse genetics systems’ have been developed in the last few decades for different classes of RNA viruses. This system enables the rapid and facile introduction of mutations and deletions and the insertion of a transgene transcriptional unit, thereby enabling the changing of the biological properties of the virus.

Reverse genetics systems for several NDV strains, including lentogenic as well as velogenic strains, were developed by the Central Veterinary Institute (CVI), part of Wageningen University and Research, currently Wageningen Bioveterinary Research (WBVR) under the supervision of Dr. Ben Peeters (Peeters et al., 1999, J. Virol. 73:5001-9; de Leeuw et al., 2005, J. Gen. Virol. 86:1759-69; Dortmans et al., 2009, J. Gen. Virol. 90:2746-50). In order to generate a reverse genetics system for providing the NDV nucleic acids and strains according to the present invention, a similar approach was used. Details of the procedure can be found in the above cited papers and in the paragraphs below. Briefly, the system consists of 4 components, i.e., a transcription plasmid containing the full-length (either authentic or genetically modified) cDNA of the virus, which is used to generate the vRNA, and 3 expression plasmids (‘helper plasmids’) containing the NP, P and L genes of NDV respectively, which are used to generate the vRNA-replication complex (consisting of NP, P and L proteins). Transcription of the cDNA (i.e. conversion of the cDNA into vRNA) and expression of the NP, P and L genes by the helper plasmids is driven by a T7 promoter. The corresponding T7 DNA-dependent RNA polymerase (T7-RNAPol) is provided by a helper-virus (Fowlpox-T7).

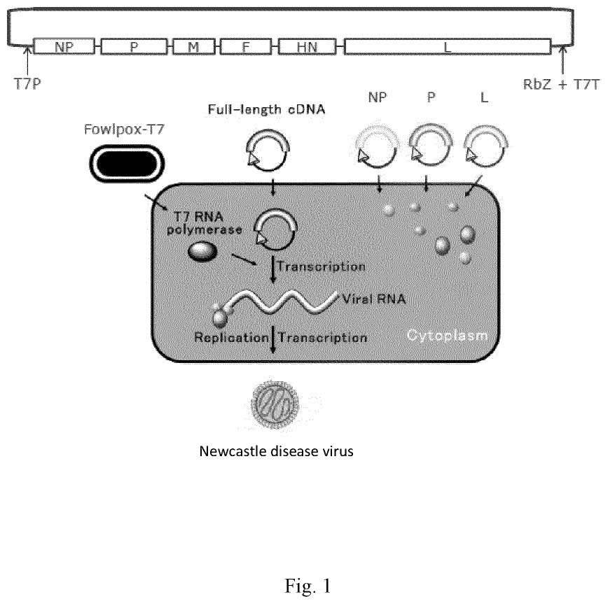

In order to rescue virus, the 4 plasmids are co-transfected into Fowlpox-T7 infected cells (FIG. 1). Three to five days after transfection, the supernatant is inoculated into specific-pathogen-free embryonated chicken eggs (ECE) and incubated for 3 days. Infectious virus that is produced by transfected cells will replicate in the ECE and progeny virus can be harvested from the allantois fluid.

In order to develop a reverse genetics system for NDV the following steps were followed:
Generation of sub-genomic NDV and foreign gene cDNA's by RT-PCR
Assembly of full-length cDNA in a transcription vector
Cloning of each of the NP, P and L genes into an expression vector
Verify nucleotide sequence of full-length cDNA and helper-plasmids
Repair nucleotide differences resulting from the cloning procedure, if necessary
Rescue of infectious virus from cDNA using co-transfection (FIG. 1)

2.2 Construction of Full-Length NDV-Mut HN(F277L)/M(G165W) cDNA and Helper Plasmids

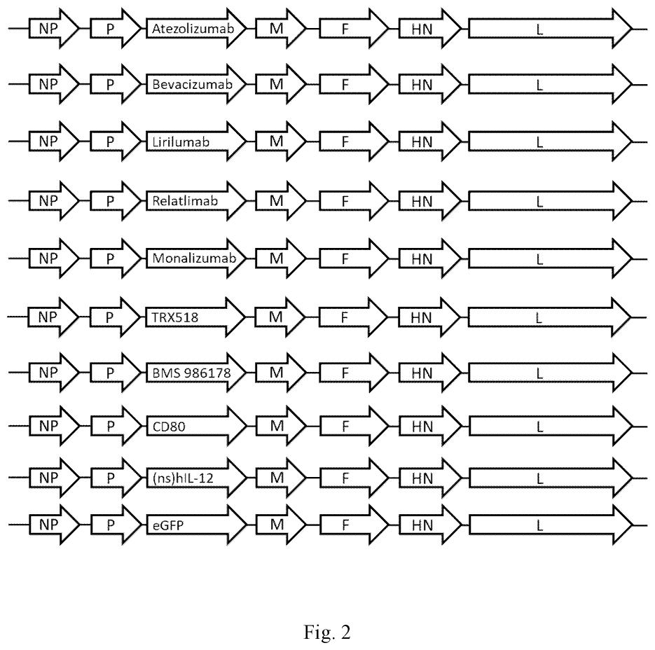

NDV-Mut HN(F277L)/M(G165W) (passage 28 HeLa cells) was used for the isolation of vRNA using standard procedures. The vRNA was used to generate first-strand cDNA by means of Reverse Transcriptase followed by PCR to generate 4 sub-genomic cDNA fragments (designated C1, C2, C3 and C8). The full-length cDNA of NDV-MutHu was assembled from these fragments and cloned in the transcription vector pOLTV5 (Peeters et al., 1999, J. Virol. 73:5001-5009) by a combination of In-Fusion® cloning and classical cloning using restriction enzymes. An overview of the procedure is shown in FIG. 2, and further details can be found in Appendix 1 of this reference. The NP, P and L-genes of NDV-MutHu were obtained by RT-PCR (Appendix 1) and cloned in the expression plasmid pCVI which was derived by deletion of a ClaI restriction fragment from pCI-neo (Promega).

2.3 Nucleotide Sequence Analysis

Nucleotide sequence analysis was used to verify that the sequence of pFL-NDV Mut HN(F277L)/M(G165W) was correct. A few nucleotides which differed from the Reference sequence were repaired. Silent mutations (i.e., not leading to an amino acid change) may be left unchanged.

2.4 Rescue of Infectious Virus from pFL-NDV Mut HN(F277L)/M(G165W)

In order to generate infectious virus, we used the co-transfection system described above (and illustrated in FIG. 1) to transfect QM5 cells (derived from Quail) using plasmid pFL-NDV_Mut HN(F277L)/M(G165W) and the helper plasmids pCVI-NP^{Mut HN(F277L)/M(G165W)}, pCVI-P^{Mut HN(F277L)/M(G165W)} and pCVI-L^{Mut HN(F277L)/M(G165W)}. Rescue of infectious virus was successful as demonstrated by the presence of infectious virus in the allantoic fluid of ECE that were inoculated with the transfection supernatant (data not shown). The identity of the rescued virus was determined by RT-PCR followed by sequencing. The rescued virus was designated rgMut HN(F277L)/M(G165W). NDV-Mut HN(F277L)/M(G165W) and rgMut HN(F277L)/M(G165W) have similar growth kinetics in HeLa cells.

### Example 3

### Identify Whether One or Both of the Amino Acid Substitutions in Mut HN(F277L)/M(G165W) are Responsible for the Difference in Growth Kinetics Between Mut HN(F277L)/M(G165W) and the Parent Strain MTH68

3.1 Growth Kinetics in HeLa Cells

The rescued rg-viruses (Table 1) as well as the original Mut HN(F277L)/M(G165W) and MTH68 viruses were used to determine their growth-kinetics in HeLa cells. Briefly, 4×10⁶ HeLa cells were seeded in 25 cm² cell culture flasks and grown overnight. The cells were infected using a MOI of 0.01 (i.e., 1 infectious virus particle per 100 cells), and at 8, 24 and 48 hours after infection the virus titer in the supernatant was determined by end-point titration on QM5 cells.

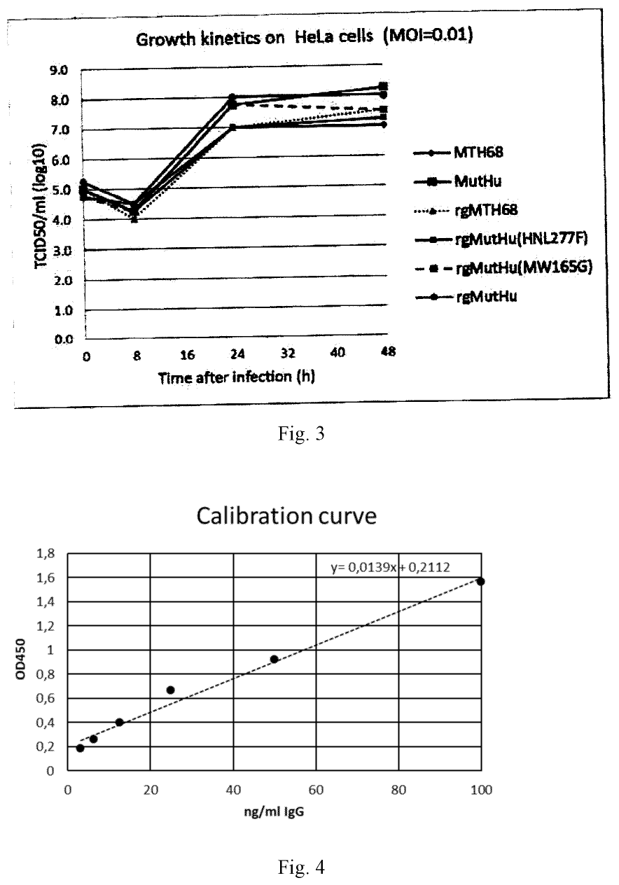

The data (FIG. 3) indicate that strains Mut HN(F277L)/M(G165W) and rgMut HN(F277L)/M(G165W) yield at least 10-fold higher virus titers than MTH68. Furthermore, the data indicate that the mutation at amino acid position 277 in the HN gene is responsible for this effect. The M mutation does not seem to have an effect. This can be best seen when looking at the virus titers 24 h after infection, or even better when comparing the increase in virus titer between 8 h and 24 h (the exponential growth phase). The virus titer shows an increase of 3.5 (log 10) for Mut HN(F277L)/M(G165W), rgMut HN(F277L)/M(G165W) and rgMut HN(F277L)/M(W165G), whereas this is 2.5 for MTH68, 2.7 for rgMut HN(L277F) and 3.0 for rgMTH68 (Table 3).

rgMut HN(F277L)/M(W165G) is a strain in which the mutation in the M gene has been restored in accordance with the NDV MTH-68/H.

TABLE 1
Virus titers (log10 TCID50/ml)

| | Time after infection (h) | | | |
|---|---|---|---|---|
| Virus | 0 h | 8 h | 24 h | 48 h |
| MTH68 | 4.8 | 4.5 | 7.0 | 7.0 |
| Mut HN(F277L)/ | 5.0 | 4.3 | 7.8 | 8.3 |
| M(G165W) | | | | |
| rgMTH68 | 5.0 | 4.0 | 7.0 | 7.5 |
| rgMut HN(L277F) | 5.0 | 4.3 | 7.0 | 7.3 |
| rgMut M(W165G) | 4.8 | 4.3 | 7.8 | 7.5 |
| rgMutHu | 5.3 | 4.5 | 8.0 | 8.0 |

### Example 4

### Generation of Recombinant NDV-Strains with Enhanced Oncolytic and Immune Stimulating Properties Due to the Expression of Different Therapeutic Proteins

To this end, three different rgMut HN(F277L)/M(G165W)/F(F117S)/F(F190L)/L(V757I)/L(F1551S)/L(R1700L) strains were generated, expressing the genes for:

1) Atezolizumab

2) Bevacizumab

4.1 Generation of Recombinant Viruses

Recombinant NDV-Mut HN(F277L)/M(G165W)/F(F117S)/F(F190L)/L(V7571)/L(F1551S)/L(R1700L) viruses (rgNDV-Mut HN(F277L)/M(G165W/F(F117S)/F(F190L)/L(V757I)/L(F1551S)/L(R1700L)) expressing Atezolizumab or Bevacizumab were generated by means of the previously established reverse genetics system described above. The genes encoding the heavy- and light-chain of Atezolizumab or the genes encoding the heavy- and light-chain of Bevacizumab were inserted into the full-length cDNA of NDV-Mut HN(F277L)/M(G165W)/F(F117S)/F(F190L)/L(V757I)/L(F1551S)/L(R1700L) between the P and the M genes. To this end the open reading frames of the foreign genes were fused via a 2A sequence. The genes were provided with the necessary NDV gene-start and gene-end sequences in order to allow transcription by the vRNA polymerase.

FIG. 2 shows the final constellation of the recombinant virus that was rescued from cloned full-length cDNA by means of the NDV reverse-genetics systems.

Infectious virus was rescued for both constructs, and virus stocks were prepared by two passages in HeLa cells. The nucleotide sequences of the inserted genes in the different recombinant viruses were verified by means of nucleotide sequence analysis and found to be correct.

Expression of Atezolizumab and Bevacizumab was determined and quantified by using a total human IgG ELISA (Invitrogen). The amount of Atezolizumab and Bevacizumab, respectively that is secreted into the medium of HeLa cells infected with the above mentioned recombinant NDV strains encoding Atezolizumab or Bevacizumab was determined by analyzing the culture supernatant of the 2^{nd} passage on HeLa cells.

As can be seen from the OD450 values given in Table 2 below, the production of Atezolizumab reached approximately 15.3 μg/ml and the production of Bevacizumab reached approximately 155.7 μg/ml.

TABLE 2
OD450 values of a human IgG ELISA.
As negative control the respectively mutated
NDV-strain without an inserted foreign gene has been used.

| | | | rgMutHu2- | rgMutHu2- | |
|---|---|---|---|---|---|
| Calibration | | Negative | Ate- | Beva- | |
| IgG ng/ml | | control | zolizumab | cizumab | Dilution |
| 100 | 1.565 | 0.071 | 2.192 | 3.51 | 1:5 |
| 50 | 0.923 | 0.081 | 1.657 | 3.441 | 1:50 |
| 25 | 0.665 | 0.079 | 0.636 | 1.573 | 1:500 |
| 12.5 | 0.401 | 0.078 | 0.307 | 0.644 | 1:5000 |
| 6.25 | 0.258 | | | | |
| 3.12 | 0.186 | | | | |
| 0 | 0.081 | | | | |
| 0 | 0.082 | | | | |

### REFERENCES

Blach-Olszewska et al., (1977) Why HeLa cells do not produce interferon? Arch Immunol Ther Exp (Warsz) 25:683-91.

Chng et al., (2015) Cleavage efficient 2A peptides for high level monoclonal antibody expression in CHO cells, mAbs, 7:2, 403-412; http://dx.doi.org/10.1080/19420862.2015.1008351.

Enoch et al., (1986) Activation of the Human beta-Interferon Gene Requires an Interferon-Inducible Factor, Mol. Cell. Biol. 6:801-10.

Haryadi et al., (2015) Optimization of Heavy Chain and Light Chain Signal Peptides for High Level Expression of Therapeutic Antibodies in CHO Cells. PLoS ONE 10(2): e0116878. doi:10.1371/journal.pone.0116878.

Schirrmacher, (2015) Oncolytic Newcastle disease virus as a prospective anti-cancer therapy. A biologic agent with potential to break therapy resistance. Expert Opin. Biol. Ther. 15:17 57-71

Zamarin et al., (2014) Localized oncolytic virotherapy overcomes systemic tumor resistance to immune checkpoint blockade immunotherapy. Sci. Transl. Med. 6(226).

Zamarin & Palese, (2017) Oncolytic Newcastle Disease Virus for cancer therapy: old challenges and new directions. Future Microbiol. 7: 347-67.

APPENDIX 1
primers used for the generation of cDNA fragments and helper-plasmids
cDNA fragments

| Fragment | Size | Primer | Sequence (5’-3’) |
|---|---|---|---|
| C1 | 3.6 kb | Noss-09 | ACGACTCACTATAGGaccaaacagagaatccgtgag |
| | | (SEQ ID | |
| | | No. 28) | |
| | | Noss-121 | CCGGGAAGATCCAGGgcactcttcttgcatgttac |
| | | (SEQ ID | |
| | | No. 29) | |
| C2 | 3.7 kb | Noss-122 | GGGCCTGCCTCACTAtggtggtaacatgcaagaag |
| | | (SEQ ID | |
| | | No. 30) | |
| | | Noss-123 | TGCATGTTACCACCAatgtgtcattgtatcgcttg |
| | | (SEQ ID | |
| | | No. 31) | |
| C3 | 5.7 kb | Noss-125 | CAAGAAGGGAGATACgtaatatacaagcgatacaatg |
| | | (SEQ ID | |
| | | No. 32) | |
| | | Noss-126 | TCGCTTGTATATTACttgttgtagcaaagagcacc |
| | | (SEQ ID | |
| | | No. 33) | |
| C8 | 2.0 kb | Noss-133 | GGCCTGGATCTTCCCattatgctgtctgtatacggtgc |
| | | (SEQ ID | |
| | | No. 34) | |
| | | Noss-10 | ATGCCATGCCGACCCaccaaacaaagacttggtgaatg |
| | | (SEQ ID | |
| | | No. 35) | |
| iPCR | 2.5 kb | Noss-17 | CCTATAGTGAGTCGTATTAATTTC |
| pOLTV5 | | (SEQ ID | |
| (StuI) | | No. 36) | |
| | | Noss-128 | CCTGGATCTTCCCGGGTCGG |
| | | (SEQ ID | |
| | | No. 37) | |
| iPCR | 2.5 kb | Noss-137 | GGGTCGGCATGGCATCTCCACC |
| pOLTV5 | | (SEQ ID | |
| (SmaI) | | No. 38) | |
| | | Noss-138 | GGGAAGATCCAGGCCTATAGTG |
| | | (SEQ ID | |
| | | No. 39) | |

Helper-plasmides (generated by In-Fusion ® cloning in pCVI)

| Gene | primer | sequence |
|---|---|---|
| NP | Noss-22 | CTCTAGAGTCGACCCttctgccaacatgtcttccg |
| | (SEQ ID | |
| | No. 40) | |
| | Noss-23 | GGGAAGCGGCCGCCCgtcggtcagtatccccagtc |
| | (SEQ ID | |
| | No. 41) | |
| P | Noss-24 | CTCTAGAGTCGACCCcagagtgaagatggccaccttc |
| | (SEQ ID | |
| | No. 42) | |
| | Noss- | GGGAAGCGGCCGCCCgtagtagtgatcagccattc |
| | 25n | |
| | (SEQ ID | |
| | No. 43) | |
| L | Noss-26 | CTCTAGAGTCGACCCgggtaggacatggcgggctc |
| | (SEQ ID | |
| | No. 44) | |
| | Noss-27 | GGGAAGCGGCCGCCCtgcctttaagagtcacagttac |
| | (SEQ ID | |
| | No. 45) | |

What is claimed is:

1. A recombinant Newcastle Disease Virus (NDV), derived from NDV strain MTH-68/H according to SEQ ID No. 1, wherein the recombinant NDV carries as a foreign gene at least one gene selected from the group consisting of
a gene encoding an antibody directed to protein PD-L1 or an antigen-binding part directed to protein PD-L1 (anti-PD-L1),
a gene encoding an antibody directed to the growth factor protein VEGF-A or an antigen-binding part directed to the growth factor protein VEGF-A (anti-VEGF-A),
a gene encoding an antibody directed to killer-cell immunoglobulin-like receptors (KIRs) or an antigen-binding part directed to killer-cell immunoglobulin-like receptors (KIRs) (anti-KIR),
a gene encoding an antibody directed to and inhibiting LAG-3 or an antigen-binding part directed to and inhibiting LAG-3 (anti-LAG-3),
a gene encoding an antibody directed to NKG2A or an antigen-binding part directed to NKG2A (anti-NKG2A),
a gene encoding an antibody directed to the glucocorticoid-induced TNF-superfamily receptor (GITR) or an antigen-binding part directed to the glucocorticoid-induced TNF-superfamily receptor (GITR) (anti-GITR),
a gene encoding an antibody directed to and activating the protein OX40 or an antigen-binding part directed to and activating the protein OX40 (anti-OX40),
a gene encoding a membrane protein which is the receptor for the proteins CD28 and CTLA-4, and which membrane protein is involved in the costimulatory signal essential for T-lymphocyte activation or a part of the membrane protein which is the receptor for the proteins CD28 and CTLA-4, and which membrane protein is involved in the costimulatory signal essential for T-lymphocyte activation,
a gene encoding a human interleukin 12 (hIL-12) or a part of a human interleukin 12 (hIL-12),
a gene encoding a green fluorescent protein or a part of a green fluorescent protein (GFP), and
any combination of these genes, parts or variants, wherein
said recombinant NDV comprises a mutated hemagglutinin-neuramidase (HN) gene and the encoded hemagglutinin-neuramidase, wherein phenylalanine (F) is substituted to leucine (L) at position 277 of the encoded HN.

2. The recombinant NDV according to claim 1, wherein the NDV further comprises a mutation in the M gene and the thus encoded matrix protein.

3. The recombinant NDV according to claim 2, wherein the matrix protein encoded by the mutated M gene has an amino acid substitution at position 165 to an amino acid with an aromatic side chain.

4. The recombinant NDV according to claim 2, wherein glycine (G) is substituted to tryptophane (W) at position 165 of the matrix protein encoded by the mutated M gene.

5. The recombinant NDV according to claim 1, wherein the recombinant NDV is encoded by and/or comprises a viral genome having a nucleic acid sequence with a sequence identity of at least 75% to the nucleic acid sequence of SEQ ID No. 1 of the sequence listing, wherein the sequence identity is determined after best alignment of the sequence of interest with the respective reference sequence.

6. The recombinant NDV according to claim 1, wherein the recombinant NDV is encoded by and/or comprises at least one of the nucleic acids according to SEQ ID No. 2, SEQ ID NO. 4 to 13 or variants thereof, variants having a sequence identity of at least 75% to the nucleic acid sequence of any one of SEQ. ID. 2 and SEQ. ID NO. 4 to 13, wherein the sequence identity is determined after best alignment of the sequence of interest with the respective reference sequence.

7. A method of treating cancer in a mammal, the method comprising administering to the mammal the recombinant NDV according to claim 1.

8. The method of claim 7, wherein the cancer is selected from the group consisting of brain tumors, bone tumors, soft tissue tumors, gynecological tumors, gastrointestinal tumors, pancreas tumors, prostate tumors, lung tumors, ear tumors, nose tumors, throat tumors, tongue tumors, and skin tumors.

9. A nucleic acid encoding a recombinant Newcastle Disease Virus (NDV), derived from NDV strain MTH-68/H according to SEQ ID No. 1, the nucleic acid comprising a transgenic construct, wherein said transgenic construct encodes a protein selected from the group consisting of
Atezolizumab, an antigen-binding part of Atezolizumab, a variant of Atezolizumab or a variant of an antigen-binding part of Atezolizumab,
Bevacizumab, an antigen-binding part of Bevacizumab, a variant of Bevacizumab or a variant of an antigen-binding part of Bevacizumab,
Lirilumab, an antigen-binding part of Lirilumab, a variant of Lirilumab or a variant of an antigen-binding part of Lirilumab,
Relatlimab, an antigen-binding part of Relatlimab, a variant of Relatlimab or a variant of an antigen-binding part of Relatlimab,
Monalizumab, an antigen-binding part of Monalizumab, a variant of Monalizumab or a variant of an antigen-binding part of Monalizumab,
TRX518, an antigen-binding part of TRX518, a variant of TRX518 or a variant of an antigen-binding part of TRX518,
BMS 986178, an antigen-binding part of BMS 986178, a variant of BMS 986178 or a variant of an antigen-binding part of BMS 986178,
CD80, a part of CD80, a variant of CD80 or a variant of a part of CD80,
a human interleukin 12 (hIL-12) or a part of a human interleukin 12 (hIL-12),
a green fluorescent protein or a part of a green fluorescent protein, and
any combination of these proteins, parts or variants, wherein
the nucleic acid in addition has a mutation in the HN gene, where the mutated HN gene encodes HN^{F277L}.

10. The nucleic acid according to claim 9, wherein the sequence encoding the recombinant NDV further comprises a mutation in the M gene, wherein the mutated M gene encodes a matrix protein M with an amino acid substitution at position 165, namely M^{G165W}.

11. (canceled)

12. The nucleic acid according to claim 9, wherein the nucleic acid consists of or comprises a nucleic acid sequence with a sequence identity of at least 75% to the nucleic acid sequence of SEQ ID No. 1 of the sequence listing, wherein the sequence identity is determined after best alignment of the sequence of interest with the respective reference sequence.

13. The nucleic acid according to claim 9, wherein the nucleic acid consists of or comprises at least one of the nucleic acids according to SEQ ID No. 2, SEQ ID No. 4 to 13 or variants thereof, the variants having a sequence identity of at least 75% to the nucleic acid sequence of any one of SEQ. ID. 2 and SEQ. ID NO. 4 to 13, wherein the sequence identity is determined after best alignment of the sequence of interest with the respective reference sequence.

14. A pharmaceutical formulation comprising particles of the recombinant NDV according to claim 1.

15. A method of oncological treatment in a mammal, the method comprising administering to the mammal the pharmaceutical formulation according to claim 14.

16. The method according to claim 14 wherein the cancer is selected from the group consisting of brain tumors, bone tumors, soft tissue tumors, gynecological tumors, gastrointestinal tumors, pancreas tumors, prostate tumors, lung tumors, ear tumors, nose tumors, throat tumors, tongue tumors, and skin tumors.

17. A pharmaceutical formulation comprising the nucleic acid according to claim 9.

18. A method of oncological treatment in a mammal, the method comprising administering to the mammal the pharmaceutical formulation according to claim 17.

19. The method according to claim 18, wherein the cancer is selected from the group consisting of brain tumors, bone tumors, soft tissue tumors, gynecological tumors, gastrointestinal tumors, pancreas tumors, prostate tumors, lung tumors, ear tumors, nose tumors, throat tumors, tongue tumors, and skin tumors.

20. The method according to claim 7, wherein the method comprises administering to the subject a combination of recombinants NDVs carring different foreign genes selected from the group consisting of:
the gene encoding the antibody directed to protein PD-L1 or the antigen-binding part directed to protein PD-L1 (anti-PD-L1), which gene encodes Atezolizumab, an antigen-binding part of Atezolizumab, a variant of Atezolizumab or a variant of an antigen-binding part of Atezolizumab,
the gene encoding the antibody directed to the growth factor protein VEGF-A or the antigen-binding part directed to the growth factor protein VEGF-A (anti-VEGF-A), which gene encodes Bevacizumab, an antigen-binding part of Bevacizumab, a variant of Bevacizumab or a variant of an antigen-binding part of Bevacizumab,
the gene encoding the antibody directed to killer-cell immunoglobulin-like receptors (KIRs) or the antigen-binding part directed to killer-cell immunoglobulin-like receptors (KIRs) (anti-KIR), which gene encodes Lirilumab, an antigen-binding part of Lirilumab, a variant of Lirilumab or a variant of an antigen-binding part of Lirilumab,
the gene encoding the antibody directed to and inhibiting LAG-3 or the antigen-binding part directed to and inhibiting LAG-3 (anti-LAG-3), which gene encodes Relatlimab, an antigen-binding part of Relatlimab, a variant of Relatlimab or a variant of an antigen-binding part of Relatlimab,
the gene encoding the antibody directed to NKG2A or the antigen-binding part directed to NKG2A (anti-NKG2A), which gene encodes Monalizumab, an antigen-binding part of Monalizumab, a variant of Monalizumab or a variant of an antigen-binding part of Monalizumab,
the gene encoding the antibody directed to the glucocorticoid-induced TNF-superfamily receptor (GITR) or the antigen-binding part directed to the glucocorticoid-induced TNF-superfamily receptor (GITR) (anti-GITR), which gene encodes TRX518, an antigen-binding part of TRX518, a variant of TRX518 or a variant of an antigen-binding part of TRX518,
the gene encoding the antibody directed to and activating the protein OX40 or the antigen-binding part directed to and activating the protein OX40 (anti-OX40), which gene encodes BMS 986178, an antigen-binding part of BMS 986178, a variant of BMS 986178 or a variant of an antigen-binding part of BMS 986178,
the gene encoding the membrane protein which is the receptor for the proteins CD28 and CTLA-4, which gene encodes CD80, a part of CD80, a variant of CD80 or a variant of a part of CD80,
the gene encoding the human interleukin 12 (hIL-12) or the part of a human interleukin 12 (hIL-12), which gene encodes non-secreting (ns)hIL-12, a part of (ns)hIL-12, a variant of (ns)hIL-12 or a variant of a part of (ns)hIL-12,
the gene encoding the green fluorescent protein or the part of a green fluorescent protein (GFP), which gene encodes enhanced GFP (eGFP), a part of eGFP, a variant of eGFP or a variant of a part of eGFP, and
any combination of these genes, parts or variants, wherein
each of said recombinant NDVs comprise the mutated hemagglutinin-neuramidase (HN) gene and the encoded hemagglutinin-neuramidase, in which encoded HN phenylalanine (F) is substituted to leucine (L) at position 277.

21. The recombinant NDV of claim 1, wherein the foreign gene is at least one gene selected from the group consisting of:
the gene encoding the antibody directed to protein PD-L1 or the antigen-binding part directed to protein PD-L1 (anti-PD-L1), which is a gene encoding Atezolizumab, an antigen-binding part of Atezolizumab, a variant of Atezolizumab or a variant of an antigen-binding part of Atezolizumab,
the gene encoding the antibody directed to the growth factor protein VEGF-A or the antigen-binding part directed to the growth factor protein VEGF-A (anti-VEGF-A), which is a gene encoding Bevacizumab, an antigen-binding part of Bevacizumab, a variant of Bevacizumab or a variant of an antigen-binding part of Bevacizumab,
the gene encoding the antibody directed to killer-cell immunoglobulin-like receptors (KIRs) or the antigen-binding part directed to killer-cell immunoglobulin-like receptors (KIRs) (anti-KIR), which is a gene encoding Lirilumab, an antigen-binding part of Lirilumab, a variant of Lirilumab or a variant of an antigen-binding part of Lirilumab,
the gene encoding the antibody directed to and inhibiting LAG-3 or the antigen-binding part directed to and inhibiting LAG-3 (anti-LAG-3), which is a gene encoding Relatlimab, an antigen-binding part of Relatlimab, a variant of Relatlimab or a variant of an antigen-binding part of Relatlimab,
the gene encoding the antibody directed to NKG2A or the antigen-binding part directed to NKG2A (anti-NKG2A), which is a gene encoding Monalizumab, an antigen-binding part of Monalizumab, a variant of Monalizumab or a variant of an antigen-binding part of Monalizumab,
the gene encoding the antibody directed to the glucocorticoid-induced TNF-superfamily receptor (GITR) or the antigen-binding part directed to the glucocorticoid-induced TNF-superfamily receptor (GITR) (anti-GITR), which is a gene encoding TRX518, an antigen-binding part of TRX518, a variant of TRX518 or a variant of an antigen-binding part of TRX518,
the gene encoding the antibody directed to and activating the protein OX40 or the antigen-binding part directed to and activating the protein OX40 (anti-OX40), which is a gene encoding BMS 986178, an antigen-binding part of BMS 986178, a variant of BMS 986178 or a variant of an antigen-binding part of BMS 986178,
the gene encoding the membrane protein which is the receptor for the proteins CD28 and CTLA-4, which is a gene encoding CD80, a part of CD80, a variant of CD80 or a variant of a part of CD80,
the gene encoding the human interleukin 12 (hIL-12) or the part of a human interleukin 12 (hIL-12), which is a gene encoding non-secreting (ns)hIL-12, a part of (ns)hIL-12, a variant of (ns)hIL-12 or a variant of a part of (ns)hIL-12,
the gene encoding the green fluorescent protein or the part of a green fluorescent protein (GFP), which is a gene encoding enhanced GFP (eGFP), a part of eGFP, a variant of eGFP or a variant of a part of eGFP, and
any combination of these genes.


SEQUENCE LISTING (45 sequences)

SEQ ID NO: 1 - DNA, 15186 bp, Newcastle disease virus
  STRAIN MTH-68/H
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatccctgggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggtttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 2 - DNA, 15186 bp, Newcastle disease virus
  Mut HN (F277L)/M(G165W)
accaaacagagaatccgtgagctacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcatccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaggggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggcatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 3 - DNA, 15186 bp, Newcastle disease virus
  Mut HN (F277L)/M(G165W)/F(F117S)/F(F190L)/L(V757I)/L(F1551S)/L(R1700L)
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 4 - DNA, 17436 bp, Newcastle disease virus
  Mut HN (F277L)/M(G165W)-Atezolizumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgcgaggtgcagctggtggagagcggcggcggcctggtgcagcccggcggcagcctgaggctgagctgcgccgccagcggcttcaccttcagcgacagctggatccactgggtgaggcaggcccccggcaagggcctggagtgggtggcctggatcagcccctacggcggcagcacctactacgccgacagcgtgaagggcaggttcaccatcagcgccgacaccagcaagaacaccgcctacctgcagatgaacagcctgagggccgaggacaccgccgtgtactactgcgccaggaggcactggcccggcggcttcgactactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacgccagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgacatccagatgacccagagccccagcagcctgagcgccagcgtgggcgacagggtgaccatcacctgcagggccagccaggacgtgagcaccgccgtggcctggtaccagcagaagcccggcaaggcccccaagctgctgatctacagcgccagcttcctgtacagcggcgtgcccagcaggttcagcggcagcggcagcggcaccgacttcaccctgaccatcagcagcctgcagcccgaggacttcgccacctactactgccagcagtacctgtaccaccccgccaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgctgataagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 5 - DNA, 17448 bp, Newcastle disease virus
  Mut HN (F277L)/M(G165W)-Bevacizumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgcgaggtgcagctggtggagagcggcggcggcctggtgcagcccggcggcagcctgaggctgagctgcgccgccagcggctacaccttcaccaactacggcatgaactgggtgaggcaggcccccggcaagggcctggagtgggtgggctggatcaacacctacaccggcgagcccacctacgccgccgacttcaagaggaggttcaccttcagcctggacaccagcaagagcaccgcctacctgcagatgaacagcctgagggccgaggacaccgccgtgtactactgcgccaagtacccccactactacggcagcagccactggtacttcgacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgacatccagatgacccagagccccagcagcctgagcgccagcgtgggcgacagggtgaccatcacctgcagcgccagccaggacatcagcaactacctgaactggtaccagcagaagcccggcaaggcccccaaggtgctgatctacttcaccagcagcctgcacagcggcgtgcccagcaggttcagcggcagcggcagcggcaccgacttcaccctgaccatcagcagcctgcagcccgaggacttcgccacctactactgccagcagtacagcaccgtgccctggaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgctgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 6 - DNA, 17442 bp, Newcastle disease virus
  Mut HN (F277L)/M(G165W)-Lirilumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgccaagtgcaactggtgcagagcggtgcagaggtgaagaagccaggatcctctgtgaaagtctcatgcaaagccagtggaggtacgttcagcttttacgcaatcagctgggtgagacaggctccgggccaaggtctggaatggatgggcggattcattccaattttcggcgcagcaaactacgcacagaagttccaggggcgggtcacgattactgctgatgagtcgacaagcacagcatacatggagctcagttctttgagaagcgatgatacagcagtttattactgtgctagaattccaagcggttcttattactatgactacgatatggatgtctggggacagggaacaacagtcaccgtatcttccgcgagcaccaagggaccttccgtgtttcccctggccccttgctccagatcaacctcagagtcgactgctgctctcggttgcttggtgaaagattattttccagaacccgttaccgtgtcttggaactccggtgcccttacttcaggggttcataccttccccgcagtcctacagtcatctggtctgtattctctcagctccgtggtcaccgtcccaagcagctcactgggcaccaagacctatacctgcaacgtcgaccataagcctagtaatacaaaggtcgacaagcgagtcgaatccaaatacggacctccttgtccaccttgccccgctcctgaattcctgggtggaccaagcgtctttctgtttccaccaaaacctaaagacacacttatgatttcccgcacacccgaagtcacctgcgtggtggtggatgtcagccaggaagaccctgaggtgcaattcaactggtacgttgatggcgtagaagtccacaacgccaagacaaaacccagagaggagcagtttaatagtacctatagggtggtgtccgtcctgaccgttctccaccaggattggttgaatggcaaggaatacaagtgtaaagtgagcaacaaaggcctaccttcgagcatagaaaagactatctccaaggcaaagggacagccaagggagccccaggtgtacacgctgccaccctctcaggaggagatgactaagaaccaggtgtccttgacctgcctcgtaaaggggttttacccctccgacatagccgtcgaatgggagagtaacggacaacccgagaacaattataaaactacacctcccgtgctcgactctgacggatccttcttcctgtactctaggctgacagtggataaatcccgttggcaggaaggaaatgtattttcctgtagtgtgatgcacgaggctctgcacaatcattatacacagaaatcactgtccttaagcctcggcaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgaaatcgtcctcacacagagcccggttacgctttcattgtctccaggcgagcgcgctaccctcagctgtagggcgtctcagtccgtttccagctatttagcttggtaccagcaaaaacctggccaggcccctagactgcttatttacgacgcctctaaccgagcgactggtatacccgcaaggttcagcggctcgggctcaggcaccgactttactctcaccatttcatcactggagcctgaggacttcgccgtgtattactgtcagcagaggagtaactggatgtacaccttcggccaagggaccaaattggaaatcaaaaggacagtggccgcgccaagcgtgtttatatttcccccctccgacgaacagttaaagagtggaaccgcatctgtggtatgtctgcttaacaatttttaccctcgcgaggcaaaggttcaatggaaagtcgacaacgccttacagtctggtaacagtcaagagtctgttacggagcaagacagcaaggactccacctattcgctgtcttcaacactaaccctcagcaaggctgattacgagaagcacaaagtttacgcctgtgaggtcacccatcaggggctttctagccctgtcacaaagtcgttcaacaggggagagtgctaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 7 - DNA, 17430 bp, Newcastle disease virus
  Mut HN (F277L)/M(G165W)-Relatlimab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgccaggtgcaactgcagcagtggggggccggactcctgaaaccatccgaaactctctcattgacttgcgccgtatacggcgggtcattttccgattactactggaattggatccgtcagcctccaggtaaaggacttgaatggataggtgaaataaatcatcgtggaagtactaacagcaatcctagtctgaaatcaagagtaacgctgagtttggacacatctaagaaccagttttcccttaaacttagaagtgttaccgctgctgacacagctgtatactactgtgctttcggttactccgattatgagtacaattggttcgacccttggggacagggcacactagtgacagtgtccagtgcttcaaccaaggggccaagcgtgtttcccttggccccctgttcacgctccactagcgaaagcacagccgcgttaggctgtctggtgaaagactattttccagagccagtcacggtgtcttggaactcgggtgccctgacatcaggtgttcatactttccccgctgtcctccagtccagtgggctgtactcattatcttcggtggtgactgtcccctcctcatcgctggggacgaagacctatacctgtaacgtggaccacaaaccaagcaacactaaggtggacaagcgcgtggaatccaaatacgggcccccatgcccaccgtgccccgcacctgagttcctgggcggtcctagcgtctttttgttcccaccgaaaccaaaggataccctcatgatttccaggacgccggaggtcacctgcgtggtcgtggacgtgagtcaggaggaccccgaagtgcaatttaactggtatgtggacggtgtggaagtccataatgcaaagaccaagcctagagaggaacaatttaatagcacgtaccgggtggttagtgtccttaccgtcttgcaccaggattggctgaacgggaaggagtataagtgcaaggtgtcaaacaagggactgccctcatcaatcgagaagacaataagtaaggcgaagggccagccacgggagcctcaggtctacacattgccgccaagtcaagaggagatgaccaaaaaccaggtgtctttgacttgcctcgtgaagggtttttacccaagtgacatcgccgtagagtgggagtctaacggccagccagagaataactataaaacgacacctccagtgcttgattctgatggatcttttttcttgtactcaaggttgaccgtggataagagcaggtggcaagaaggcaacgtttttagttgttccgttatgcacgaggccttgcataatcactatactcagaagtccctatccctgtctctcgggcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgaaattgtgctgacacaatcgccagccactctgtctctgagccctggtgagcgggccactctgtcctgcagggccagtcagtctatttccagctacttggcttggtatcagcaaaaacccggccaagctcctagattgcttatttacgacgcgagcaatagggccaccggtattccagctcgctttagcggctccggaagtggtactgatttcacgctgacaatttcaagcttggaacccgaggattttgcagtgtattactgccagcagagaagcaattggccgctgaccttcggccaggggactaatcttgaaataaaacgcacggtggctgccccctccgtcttcatctttccaccttctgacgagcaattgaagagcggtaccgctagcgtagtgtgtcttctgaacaatttctacccacgcgaggccaaagtgcaatggaaagtcgacaatgctctgcagtcaggaaactcccaggagagcgtcacagagcaagattctaaagattccacttacagtctgtcatcgacattgacactgagcaaggccgattacgaaaagcataaagtgtacgcttgtgaggtcactcaccaaggactgagctcaccagttaccaaatcgtttaatagaggtgagtgttaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 8 - DNA, 17448 bp, Newcastle disease virus
  Mut HN (F277L)/M(G165W)-Monalizumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgccaggttcagttggtacagtctggcgcggaggtgaaaaaacctggagcatctgtcaaggtaagttgtaaagcttcgggctatacctttacctcatattggatgaactgggtgcggcaggcccccggtcaagggcttgaatggatgggcagaattgatccttatgattccgaaacacattacgctcagaagctgcagggcagggttaccatgacgacagacaccagcacttccactgcttacatggagctgcgatctctgcggtcagatgacacggctgtgtactattgcgccagaggcggatacgattttgacgtggggactctgtattggttctttgacgtctgggggcagggaaccactgtgacagtctcatcggcgtcaactaaagggccaagcgtgttccccttggcaccatgtagtcggtccaccagtgaaagtaccgctgcactcggttgtctggtaaaagattattttccagagcccgttactgtaagctggaacagtggggccttgacgtctggagtccatacctttccggcggtccttcagtcatctgggctgtacagcctttccagcgttgtcactgttccaagctcctccctgggcacgaagacctatacatgtaatgtcgaccataagccttcaaatacaaaagtagacaaacgtgtggaatcaaaatacggacccccatgcccgccctgtccagcgcctgaattcttgggaggcccgtctgtctttttattccccccgaaaccgaaggacaccctcatgatctcaaggacaccagaggtgacctgcgtggtggtggatgttagtcaagaggatccagaggtgcagtttaactggtatgtcgatggcgtcgaagtccacaacgctaaaaccaagccacgggaggagcagtttaactctacctatcgagtggtgtccgtactgactgtgttgcaccaggactggcttaatgggaaagaatacaagtgtaaagtttcaaacaaaggcctgccttcaagtatcgaaaagactattagcaaagctaaagggcagccaagagaaccacaggtgtacactttgcccccttcccaagaggagatgactaagaatcaagtttctctgacatgtctggtaaaaggcttctacccttctgatatagcagtggagtgggagtctaacggacagcccgaaaacaattacaagacgacaccccctgtgctagattcagacggctctttttttctttacagtcggctcacagtggataaatcacgctggcaggaagggaatgtctttagctgcagcgtcatgcatgaggctttgcacaaccattacactcagaaatctctgagcctctcacttggtaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgatatccagatgacccagagcccctccagccttagcgcttcagtcggggatagagtgacaattacctgcagagcgtctgaaaatatctacagctacttggcctggtaccaacagaaaccaggcaaagctcctaagctcctgatttataacgcaaaaaccttggctgagggggtgcccagtcgcttctcgggctctggctcaggaaccgactttacactcaccatttcttcactgcagcctgaggacttcgctacgtactactgccagcatcactatggtaccccacgcacttttggaggggggactaaggtggaaatcaagaggaccgtggccgccccatccgtatttatcttccccccatccgatgagcagttaaaatcagggaccgcctcggttgtgtgtctgcttaacaacttttacccccgcgaagctaaggtgcagtggaaggttgataacgccttgcagtctggaaacagccaggagagcgtcacagaacaagattcaaaagacagtacttattccctgagtagtacgctgaccctgtccaaagccgattacgaaaagcacaaagtatacgcgtgtgaggtcactcaccagggactatcaagcccggtcactaaatcattcaacaggggggagtgctaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 9 - DNA, 17436 bp, Newcastle disease virus
  Mut HN (F277L)/M(G165W)-TRX518
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgccaagtgaccttacgggaatcaggtccagcgctggtaaagcccacccaaactctaacattgacctgtactttcagcggcttttccctttcgacaagtggcatgggggtgggatggatccggcaacctcctgggaaggccctcgaatggctggcccacatttggtgggacgatgacaagtactacaacccctcactgaagtcacggctgacgatttcaaaggacaccagcaaaaatcaagtggtactgacgatgaccaatatggatcccgtcgacactgctacatactactgcgctcgcacaaggcgatactttcccttcgcctattggggacagggaactctggttaccgtgagttccgcatccactaagggcccaagcgtcttcccgctggccccttccagtaagagcacctccggagggaccgctgcacttggatgcctggttaaagactattttcctgaacctgttacagtcagttggaactcgggggcactaacctctggagtgcataccttcccagctgtactccaatcatctggactatactcactttcgtctgtcgtgactgtgccctcttcttcacttgggacccagacatatatttgcaacgtcaatcataagccatcgaacaccaaggtggataagaaggtggagccgaaatcttgtgataagacccacacatgtcctccatgcccagcccctgagctgttgggaggtccctctgtgttcctgttccccccaaaaccaaaagacaccctcatgatctccagaacgcctgaggtcacctgtgtcgttgtggacgtttctcacgaagacccagaggtcaaattcaattggtatgtcgacggggtagaggtccacaatgctaagaccaagcctcgagaagagcaatatgcttccacttacagagtggtgtctgttctgaccgtcctgcaccaagactggctaaatggaaaagagtataaatgcaaggtcagcaacaaagctctgcccgccccaattgaaaagactatcagcaaagcgaaaggccagccacgggaacctcaggtctatacgctgcccccaagccgtgatgagctcacaaagaaccaggtgagtctgacttgtctagtgaaaggcttttaccccagtgacattgcagtggagtgggagtccaacggacaacccgaaaacaactataaaactaccccccccgttctcgattctgatggcagcttcttcttatacagcaaacttacggtggataaatcgcggtggcagcagggaaatgtgttttcctgctctgtcatgcatgaggccctacataatcactatacacaaaagtctctctctttgtcccccggaaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgagatagtgatgactcagtcccccgccactctctcagtaagcccaggagagagagctacgctgagctgcaaggcctcccagaacgtggggaccaacgtagcctggtaccaacaaaagcccggtcaggccccccggctgctcatttattccgcaagttatagatattcaggcattccggccagatttagtggatccggctcaggcacggagtttaccttgaccattagctctctccaatctgaggatttcgcggtgtattattgtcagcagtataacacagaccccttgaccttcggcggtggcacaaaagtcgaaattaagaggacagtcgccgccccttccgtgttcattttccccccctcagacgaacagctgaaatctggcacagcttcgtggtgtttactgaacaacttctatcccagagaagcaaaggtccagtggaaggtcgataatgccctgcagtctggtaatagtcaggagtccgtaactgagcaggacagcaaggactccacctacagcctgtcctctaccctgacactgagcaaagcagactacgaaaaacacaaagtgtacgcctgtgaggtcactcaccaggggctgagctctccagtgactaagtcttttaatcgcggcgagtgctaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 10 - DNA, 17430 bp, Newcastle disease virus
  Mut HN (F277L)/M(G165W)-BMS 986178
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgcgaagtgcagctagtgcagagtgggggcgggctagtgcagccaggggggtctcttcgcttgtcttgcgccggtagcggttttaccttttcatcgtatgcaatgtactgggtgaggcaagcccctggcaagggattggagtgggtgagcgcgatagacactgacgccggtaccttctatgcggatagtgtaagggggcgcttcaccattagtcgcgacaacgccaagaactctctttatctccagatgaattcactccgagctgaggacaccgcagtctacttttgtgctaggctgggggaaggctacttctttgattactggggacagggaaccttggtgactgtgagctctgcctccacgaaaggtccttctgttttcccccttgccccctcctcaaaatctacctctggaggtaccgccgctctaggctgcctagtaaaggattacttccctgaacccgtgacggtgtcttggaacagtggtgctcttacaagtggcgtgcacacctttcctgccgtgctccaaagttctgggctttattctctgtcctccgtggtgaccgtgcccagcagctctcttggtacccagacttacatctgtaatgttaaccacaaaccctcaaatacaaaggtggacaagagagtggagccaaagagctgcgataaaacccacacctgtcccccctgtcccgcacctgagcttcttgggggccctagtgtgtttctgttcccacctaagcccaaagacaccctgatgatctctaggaccccggaggttacctgcgtagttgtggacgtctcccacgaggatcccgaggtgaaattcaattggtacgtcgacggcgttgaagtccataacgccaagaccaagcctcgcgaagaacagtataactctacctatcgcgtggtgtcggtcctgactgtgcttcaccaggactggcttaacgggaaggaatataaatgcaaggtcagcaataaggccctccccgctccaattgagaagaccatttccaaagctaaaggccagccacgggagccccaggtctacactctaccaccctcccgcgaggagatgaccaaaaaccaggtgtccctgacctgtttggtgaagggattttacccgtccgatatcgctgtggagtgggaaagtaatggccaacctgaaaacaactataagacaaccccgccagtcctagatagcgatggatccttcttcctgtatagtaaacttacagtggataaaagtaggtggcagcagggtaacgttttttcgtgtagtgtgatgcatgaagcactccacaatcactatacccaaaaaagcctttcgctgtcccctggccgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgagattgtgctgacgcagagcccagcaactttgagtctctcgcccggcgagcgcgccacactttcctgtcgagcctcccaatccgtgagcagttacctggcatggtaccaacagaagcctgggcaagcccccagattgctgatttatgatgcctccaatcgagcaacaggaataccagctcggttcagtgggagcggaagcggtacagatttcactctgacaatcagctcgctggagccagaggacttcgctgtttattactgccagcagcgttcgaactggcccccaacgtttggaggaggcaccaaagtggaaataaaacgaacagtagcagccccatctgtcttcatcttcccccccagcgatgaacagctgaagtcaggcactgcttccgtcgtctgcctactgaacaatttttatcctagagaggccaaggtccaatggaaggtagacaacgcactccaatcagggaactcacaagagtctgtcaccgagcaggactctaaggactcgacatactcattatctagtaccctgacactgagcaaggccgactacgaaaaacataaagtctacgcctgtgaagtgacgcaccagggactcagctctcccgtcactaagagcttcaatcggggggagtgttaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 11 - DNA, 16122 bp, Newcastle disease virus
  Mut HN (F277L)/M(G165W)-CD80
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagtggctttcacttttgaccctaagcatctgccaccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 12 - DNA, 16824 bp, Newcastle disease virus
  Mut HN (F277L)/M(G165W)-(ns)hIL-12
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatgagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggctccggcgagggccgcggctccctgctgacctgcggcgacgtggaggagaaccccggccccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 13 - DNA, 15966 bp, Newcastle disease virus
  Mut HN (F277L)/M(G165W)-eGFP
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaattggagcggccgcattgtgccaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 14 - DNA, 17436 bp, Newcastle disease virus
  Mut HN (F277L)/M(G165W)/F(F117S)/F(F190L)/ L(V757I)/L(F1551S)/L(R1 700L)-Atezolizumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgcgaggtgcagctggtggagagcggcggcggcctggtgcagcccggcggcagcctgaggctgagctgcgccgccagcggcttcaccttcagcgacagctggatccactgggtgaggcaggcccccggcaagggcctggagtgggtggcctggatcagcccctacggcggcagcacctactacgccgacagcgtgaagggcaggttcaccatcagcgccgacaccagcaagaacaccgcctacctgcagatgaacagcctgagggccgaggacaccgccgtgtactactgcgccaggaggcactggcccggcggcttcgactactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacgccagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgacatccagatgacccagagccccagcagcctgagcgccagcgtgggcgacagggtgaccatcacctgcagggccagccaggacgtgagcaccgccgtggcctggtaccagcagaagcccggcaaggcccccaagctgctgatctacagcgccagcttcctgtacagcggcgtgcccagcaggttcagcggcagcggcagcggcaccgacttcaccctgaccatcagcagcctgcagcccgaggacttcgccacctactactgccagcagtacctgtaccaccccgccaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgctgataagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 15 - DNA, 17448 bp, Newcastle disease virus
  Mut HN (F277L)/M(G165W)/F(F117S)/F(F190L)/ L(V757I)/L(F1551S)/L(R1 700L)-Bevacizumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgcgaggtgcagctggtggagagcggcggcggcctggtgcagcccggcggcagcctgaggctgagctgcgccgccagcggctacaccttcaccaactacggcatgaactgggtgaggcaggcccccggcaagggcctggagtgggtgggctggatcaacacctacaccggcgagcccacctacgccgccgacttcaagaggaggttcaccttcagcctggacaccagcaagagcaccgcctacctgcagatgaacagcctgagggccgaggacaccgccgtgtactactgcgccaagtacccccactactacggcagcagccactggtacttcgacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgacatccagatgacccagagccccagcagcctgagcgccagcgtgggcgacagggtgaccatcacctgcagcgccagccaggacatcagcaactacctgaactggtaccagcagaagcccggcaaggcccccaaggtgctgatctacttcaccagcagcctgcacagcggcgtgcccagcaggttcagcggcagcggcagcggcaccgacttcaccctgaccatcagcagcctgcagcccgaggacttcgccacctactactgccagcagtacagcaccgtgccctggaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgctgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 16 - DNA, 17442 bp, Newcastle disease virus
  Mut HN (F277L)/M(G165W)/F(F117S)/F(F190L)/ L(V757I)/L(F1551S)/L(R1 700L)-Lirilumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgccaagtgcaactggtgcagagcggtgcagaggtgaagaagccaggatcctctgtgaaagtctcatgcaaagccagtggaggtacgttcagcttttacgcaatcagctgggtgagacaggctccgggccaaggtctggaatggatgggcggattcattccaattttcggcgcagcaaactacgcacagaagttccaggggcgggtcacgattactgctgatgagtcgacaagcacagcatacatggagctcagttctttgagaagcgatgatacagcagtttattactgtgctagaattccaagcggttcttattactatgactacgatatggatgtctggggacagggaacaacagtcaccgtatcttccgcgagcaccaagggaccttccgtgtttcccctggccccttgctccagatcaacctcagagtcgactgctgctctcggttgcttggtgaaagattattttccagaacccgttaccgtgtcttggaactccggtgcccttacttcaggggttcataccttccccgcagtcctacagtcatctggtctgtattctctcagctccgtggtcaccgtcccaagcagctcactgggcaccaagacctatacctgcaacgtcgaccataagcctagtaatacaaaggtcgacaagcgagtcgaatccaaatacggacctccttgtccaccttgccccgctcctgaattcctgggtggaccaagcgtctttctgtttccaccaaaacctaaagacacacttatgatttcccgcacacccgaagtcacctgcgtggtggtggatgtcagccaggaagaccctgaggtgcaattcaactggtacgttgatggcgtagaagtccacaacgccaagacaaaacccagagaggagcagtttaatagtacctatagggtggtgtccgtcctgaccgttctccaccaggattggttgaatggcaaggaatacaagtgtaaagtgagcaacaaaggcctaccttcgagcatagaaaagactatctccaaggcaaagggacagccaagggagccccaggtgtacacgctgccaccctctcaggaggagatgactaagaaccaggtgtccttgacctgcctcgtaaaggggttttacccctccgacatagccgtcgaatgggagagtaacggacaacccgagaacaattataaaactacacctcccgtgctcgactctgacggatccttcttcctgtactctaggctgacagtggataaatcccgttggcaggaaggaaatgtattttcctgtagtgtgatgcacgaggctctgcacaatcattatacacagaaatcactgtccttaagcctcggcaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgaaatcgtcctcacacagagcccggttacgctttcattgtctccaggcgagcgcgctaccctcagctgtagggcgtctcagtccgtttccagctatttagcttggtaccagcaaaaacctggccaggcccctagactgcttatttacgacgcctctaaccgagcgactggtatacccgcaaggttcagcggctcgggctcaggcaccgactttactctcaccatttcatcactggagcctgaggacttcgccgtgtattactgtcagcagaggagtaactggatgtacaccttcggccaagggaccaaattggaaatcaaaaggacagtggccgcgccaagcgtgtttatatttcccccctccgacgaacagttaaagagtggaaccgcatctgtggtatgtctgcttaacaatttttaccctcgcgaggcaaaggttcaatggaaagtcgacaacgccttacagtctggtaacagtcaagagtctgttacggagcaagacagcaaggactccacctattcgctgtcttcaacactaaccctcagcaaggctgattacgagaagcacaaagtttacgcctgtgaggtcacccatcaggggctttctagccctgtcacaaagtcgttcaacaggggagagtgctaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 17 - DNA, 17430 bp, Newcastle disease virus
  Mut HN (F277L)/M(G165W)/F(F117S)/F(F190L)/ L(V757I)/L(F1551S)/L(R1 700L)-Relatlimab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgccaggtgcaactgcagcagtggggggccggactcctgaaaccatccgaaactctctcattgacttgcgccgtatacggcgggtcattttccgattactactggaattggatccgtcagcctccaggtaaaggacttgaatggataggtgaaataaatcatcgtggaagtactaacagcaatcctagtctgaaatcaagagtaacgctgagtttggacacatctaagaaccagttttcccttaaacttagaagtgttaccgctgctgacacagctgtatactactgtgctttcggttactccgattatgagtacaattggttcgacccttggggacagggcacactagtgacagtgtccagtgcttcaaccaaggggccaagcgtgtttcccttggccccctgttcacgctccactagcgaaagcacagccgcgttaggctgtctggtgaaagactattttccagagccagtcacggtgtcttggaactcgggtgccctgacatcaggtgttcatactttccccgctgtcctccagtccagtgggctgtactcattatcttcggtggtgactgtcccctcctcatcgctggggacgaagacctatacctgtaacgtggaccacaaaccaagcaacactaaggtggacaagcgcgtggaatccaaatacgggcccccatgcccaccgtgccccgcacctgagttcctgggcggtcctagcgtctttttgttcccaccgaaaccaaaggataccctcatgatttccaggacgccggaggtcacctgcgtggtcgtggacgtgagtcaggaggaccccgaagtgcaatttaactggtatgtggacggtgtggaagtccataatgcaaagaccaagcctagagaggaacaatttaatagcacgtaccgggtggttagtgtccttaccgtcttgcaccaggattggctgaacgggaaggagtataagtgcaaggtgtcaaacaagggactgccctcatcaatcgagaagacaataagtaaggcgaagggccagccacgggagcctcaggtctacacattgccgccaagtcaagaggagatgaccaaaaaccaggtgtctttgacttgcctcgtgaagggtttttacccaagtgacatcgccgtagagtgggagtctaacggccagccagagaataactataaaacgacacctccagtgcttgattctgatggatcttttttcttgtactcaaggttgaccgtggataagagcaggtggcaagaaggcaacgtttttagttgttccgttatgcacgaggccttgcataatcactatactcagaagtccctatccctgtctctcgggcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgaaattgtgctgacacaatcgccagccactctgtctctgagccctggtgagcgggccactctgtcctgcagggccagtcagtctatttccagctacttggcttggtatcagcaaaaacccggccaagctcctagattgcttatttacgacgcgagcaatagggccaccggtattccagctcgctttagcggctccggaagtggtactgatttcacgctgacaatttcaagcttggaacccgaggattttgcagtgtattactgccagcagagaagcaattggccgctgaccttcggccaggggactaatcttgaaataaaacgcacggtggctgccccctccgtcttcatctttccaccttctgacgagcaattgaagagcggtaccgctagcgtagtgtgtcttctgaacaatttctacccacgcgaggccaaagtgcaatggaaagtcgacaatgctctgcagtcaggaaactcccaggagagcgtcacagagcaagattctaaagattccacttacagtctgtcatcgacattgacactgagcaaggccgattacgaaaagcataaagtgtacgcttgtgaggtcactcaccaaggactgagctcaccagttaccaaatcgtttaatagaggtgagtgttaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 18 - DNA, 17448 bp, Newcastle disease virus
  Mut HN (F277L)/M(G165W)/F(F117S)/F(F190L)/ L(V757I)/L(F1551S)/L(R1 700L)-Monalizumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgccaggttcagttggtacagtctggcgcggaggtgaaaaaacctggagcatctgtcaaggtaagttgtaaagcttcgggctatacctttacctcatattggatgaactgggtgcggcaggcccccggtcaagggcttgaatggatgggcagaattgatccttatgattccgaaacacattacgctcagaagctgcagggcagggttaccatgacgacagacaccagcacttccactgcttacatggagctgcgatctctgcggtcagatgacacggctgtgtactattgcgccagaggcggatacgattttgacgtggggactctgtattggttctttgacgtctgggggcagggaaccactgtgacagtctcatcggcgtcaactaaagggccaagcgtgttccccttggcaccatgtagtcggtccaccagtgaaagtaccgctgcactcggttgtctggtaaaagattattttccagagcccgttactgtaagctggaacagtggggccttgacgtctggagtccatacctttccggcggtccttcagtcatctgggctgtacagcctttccagcgttgtcactgttccaagctcctccctgggcacgaagacctatacatgtaatgtcgaccataagccttcaaatacaaaagtagacaaacgtgtggaatcaaaatacggacccccatgcccgccctgtccagcgcctgaattcttgggaggcccgtctgtctttttattccccccgaaaccgaaggacaccctcatgatctcaaggacaccagaggtgacctgcgtggtggtggatgttagtcaagaggatccagaggtgcagtttaactggtatgtcgatggcgtcgaagtccacaacgctaaaaccaagccacgggaggagcagtttaactctacctatcgagtggtgtccgtactgactgtgttgcaccaggactggcttaatgggaaagaatacaagtgtaaagtttcaaacaaaggcctgccttcaagtatcgaaaagactattagcaaagctaaagggcagccaagagaaccacaggtgtacactttgcccccttcccaagaggagatgactaagaatcaagtttctctgacatgtctggtaaaaggcttctacccttctgatatagcagtggagtgggagtctaacggacagcccgaaaacaattacaagacgacaccccctgtgctagattcagacggctctttttttctttacagtcggctcacagtggataaatcacgctggcaggaagggaatgtctttagctgcagcgtcatgcatgaggctttgcacaaccattacactcagaaatctctgagcctctcacttggtaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgatatccagatgacccagagcccctccagccttagcgcttcagtcggggatagagtgacaattacctgcagagcgtctgaaaatatctacagctacttggcctggtaccaacagaaaccaggcaaagctcctaagctcctgatttataacgcaaaaaccttggctgagggggtgcccagtcgcttctcgggctctggctcaggaaccgactttacactcaccatttcttcactgcagcctgaggacttcgctacgtactactgccagcatcactatggtaccccacgcacttttggaggggggactaaggtggaaatcaagaggaccgtggccgccccatccgtatttatcttccccccatccgatgagcagttaaaatcagggaccgcctcggttgtgtgtctgcttaacaacttttacccccgcgaagctaaggtgcagtggaaggttgataacgccttgcagtctggaaacagccaggagagcgtcacagaacaagattcaaaagacagtacttattccctgagtagtacgctgaccctgtccaaagccgattacgaaaagcacaaagtatacgcgtgtgaggtcactcaccagggactatcaagcccggtcactaaatcattcaacaggggggagtgctaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 19 - DNA, 17436 bp, Newcastle disease virus
  Mut HN (F277L)/M(G165W)/F(F117S)/F(F190L)/ L(V757I)/L(F1551S)/L(R1 700L)-TRX518
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgccaagtgaccttacgggaatcaggtccagcgctggtaaagcccacccaaactctaacattgacctgtactttcagcggcttttccctttcgacaagtggcatgggggtgggatggatccggcaacctcctgggaaggccctcgaatggctggcccacatttggtgggacgatgacaagtactacaacccctcactgaagtcacggctgacgatttcaaaggacaccagcaaaaatcaagtggtactgacgatgaccaatatggatcccgtcgacactgctacatactactgcgctcgcacaaggcgatactttcccttcgcctattggggacagggaactctggttaccgtgagttccgcatccactaagggcccaagcgtcttcccgctggccccttccagtaagagcacctccggagggaccgctgcacttggatgcctggttaaagactattttcctgaacctgttacagtcagttggaactcgggggcactaacctctggagtgcataccttcccagctgtactccaatcatctggactatactcactttcgtctgtcgtgactgtgccctcttcttcacttgggacccagacatatatttgcaacgtcaatcataagccatcgaacaccaaggtggataagaaggtggagccgaaatcttgtgataagacccacacatgtcctccatgcccagcccctgagctgttgggaggtccctctgtgttcctgttccccccaaaaccaaaagacaccctcatgatctccagaacgcctgaggtcacctgtgtcgttgtggacgtttctcacgaagacccagaggtcaaattcaattggtatgtcgacggggtagaggtccacaatgctaagaccaagcctcgagaagagcaatatgcttccacttacagagtggtgtctgttctgaccgtcctgcaccaagactggctaaatggaaaagagtataaatgcaaggtcagcaacaaagctctgcccgccccaattgaaaagactatcagcaaagcgaaaggccagccacgggaacctcaggtctatacgctgcccccaagccgtgatgagctcacaaagaaccaggtgagtctgacttgtctagtgaaaggcttttaccccagtgacattgcagtggagtgggagtccaacggacaacccgaaaacaactataaaactaccccccccgttctcgattctgatggcagcttcttcttatacagcaaacttacggtggataaatcgcggtggcagcagggaaatgtgttttcctgctctgtcatgcatgaggccctacataatcactatacacaaaagtctctctctttgtcccccggaaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgagatagtgatgactcagtcccccgccactctctcagtaagcccaggagagagagctacgctgagctgcaaggcctcccagaacgtggggaccaacgtagcctggtaccaacaaaagcccggtcaggccccccggctgctcatttattccgcaagttatagatattcaggcattccggccagatttagtggatccggctcaggcacggagtttaccttgaccattagctctctccaatctgaggatttcgcggtgtattattgtcagcagtataacacagaccccttgaccttcggcggtggcacaaaagtcgaaattaagaggacagtcgccgccccttccgtgttcattttccccccctcagacgaacagctgaaatctggcacagcttcgtggtgtttactgaacaacttctatcccagagaagcaaaggtccagtggaaggtcgataatgccctgcagtctggtaatagtcaggagtccgtaactgagcaggacagcaaggactccacctacagcctgtcctctaccctgacactgagcaaagcagactacgaaaaacacaaagtgtacgcctgtgaggtcactcaccaggggctgagctctccagtgactaagtcttttaatcgcggcgagtgctaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 20 - DNA, 17430 bp, Newcastle disease virus
  Mut HN (F277L)/M(G165W)/F(F117S)/F(F190L)/ L(V757I)/L(F1551S)/L(R1 700L)-BMS 986178
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgttccgcggcgtgcagtgcgaagtgcagctagtgcagagtgggggcgggctagtgcagccaggggggtctcttcgcttgtcttgcgccggtagcggttttaccttttcatcgtatgcaatgtactgggtgaggcaagcccctggcaagggattggagtgggtgagcgcgatagacactgacgccggtaccttctatgcggatagtgtaagggggcgcttcaccattagtcgcgacaacgccaagaactctctttatctccagatgaattcactccgagctgaggacaccgcagtctacttttgtgctaggctgggggaaggctacttctttgattactggggacagggaaccttggtgactgtgagctctgcctccacgaaaggtccttctgttttcccccttgccccctcctcaaaatctacctctggaggtaccgccgctctaggctgcctagtaaaggattacttccctgaacccgtgacggtgtcttggaacagtggtgctcttacaagtggcgtgcacacctttcctgccgtgctccaaagttctgggctttattctctgtcctccgtggtgaccgtgcccagcagctctcttggtacccagacttacatctgtaatgttaaccacaaaccctcaaatacaaaggtggacaagagagtggagccaaagagctgcgataaaacccacacctgtcccccctgtcccgcacctgagcttcttgggggccctagtgtgtttctgttcccacctaagcccaaagacaccctgatgatctctaggaccccggaggttacctgcgtagttgtggacgtctcccacgaggatcccgaggtgaaattcaattggtacgtcgacggcgttgaagtccataacgccaagaccaagcctcgcgaagaacagtataactctacctatcgcgtggtgtcggtcctgactgtgcttcaccaggactggcttaacgggaaggaatataaatgcaaggtcagcaataaggccctccccgctccaattgagaagaccatttccaaagctaaaggccagccacgggagccccaggtctacactctaccaccctcccgcgaggagatgaccaaaaaccaggtgtccctgacctgtttggtgaagggattttacccgtccgatatcgctgtggagtgggaaagtaatggccaacctgaaaacaactataagacaaccccgccagtcctagatagcgatggatccttcttcctgtatagtaaacttacagtggataaaagtaggtggcagcagggtaacgttttttcgtgtagtgtgatgcatgaagcactccacaatcactatacccaaaaaagcctttcgctgtcccctggccgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgagattgtgctgacgcagagcccagcaactttgagtctctcgcccggcgagcgcgccacactttcctgtcgagcctcccaatccgtgagcagttacctggcatggtaccaacagaagcctgggcaagcccccagattgctgatttatgatgcctccaatcgagcaacaggaataccagctcggttcagtgggagcggaagcggtacagatttcactctgacaatcagctcgctggagccagaggacttcgctgtttattactgccagcagcgttcgaactggcccccaacgtttggaggaggcaccaaagtggaaataaaacgaacagtagcagccccatctgtcttcatcttcccccccagcgatgaacagctgaagtcaggcactgcttccgtcgtctgcctactgaacaatttttatcctagagaggccaaggtccaatggaaggtagacaacgcactccaatcagggaactcacaagagtctgtcaccgagcaggactctaaggactcgacatactcattatctagtaccctgacactgagcaaggccgactacgaaaaacataaagtctacgcctgtgaagtgacgcaccagggactcagctctcccgtcactaagagcttcaatcggggggagtgttaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 21 - DNA, 16122 bp, Newcastle disease virus
  Mut HN (F277L)/M(G165W)/F(F117S)/F(F190L)/ L(V757I)/L(F1551S)/L(R1 700L)-CD80
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagtggctttcacttttgaccctaagcatctgccaccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 22 - DNA, 16824 bp, Newcastle disease virus
  Mut HN (F277L)/M(G165W)/F(F117S)/F(F190L)/ L(V757I)/L(F1551S)/L(R1 700L)-(ns)hIL-12
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatgagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggctccggcgagggccgcggctccctgctgacctgcggcgacgtggaggagaaccccggccccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 23 - DNA, 15966 bp, Newcastle disease virus
  Mut HN (F277L)/M(G165W)/F(F117S)/F(F190L)/ L(V757I)/L(F1551S)/L(R1 700L)-eGFP
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaattggagcggccgcattgtgccaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 24 - protein, 571 aa, Newcastle disease virus
  HN protein
MDSAVSQVALENDRREANNTWRLVFRIAALLLLVITLAVSAVALAYSMEASTPGDLVSIPTAISRAEERITSALGSNQDVVDRIYKQVALESPLALLNTESIIMNAITSLSYQINGATNNSGCGAPVHDPDYIGGIGQELIVDDTSDVTSFYPSAFQEHLNFIPAPTTGSGCTRIPSFDMSATHYCYTHNVILSGCRDHSHSHQYLALGVLRTSATGRVFFSTLRSINLDDAQNRKSCSVSATPLGCDMLCSKITETEEEDYKSVIPTSMVHGRLGLDGQYHEKDLDVTTLFRDWVANYPGVGGGSFINNRVWFPVYGGLKPSSPSDTAQEGRYVIYKRYNDTCPDEQDYQIRMAKSSYKPRRFGGKRVQQAILSIKVSTSLGEDPVLTVPPNTVALMGAEGRVLTVGTSHFLYQRGSSYFSPALLYPMTVNNKTATLHNPYTFNAFTRPGSVPCQASARCPNSCVTGVYTDPYPLVFHRNHTLRGVFGTMLDDEQARLNPVSAVFDNISRSRITRVSSSSTRAAYTTSTCFKVVKTNKTYCLSIAEISNTLFGEFRIVPLLVEILKDGGV

SEQ ID NO: 25 - protein, 364 aa, Newcastle disease virus
  M protein
MDSSRTIGLYFDSALPSSNLLAFPIVLRDVGDGKKQITPQYRIRRLDSWTDSKEDSVFITTYGFIFQVGNEEVTVGMINDNPKRELLSAAMLCLGSVPNVGDPVELARACLTMVVTCKKSATNTERMVFSVVQAPRVLQSCRVVADKYSSVNAVKHVKAPEKIPWSETLEYKVNFVSLTVVPKKDVYKIPTAVLKVSGSSLYNLALNVTIDVEVDPKSPLVKSLSRSDSGYYANLFLHIGLMSTVDKRGKKVTFDQLERKIRRLDLSVGLSDVLGPSVLVKARGARTRLLAPFFSNSGTACYPIANASPQVAKILWSQTACLRSVKIIIQAGTQRAVAVTADHEVTSTKIEKRHTIAKYNPFKK

SEQ ID NO: 26 - protein, 553 aa, Newcastle disease virus
  F protein
MGPRSSTRIPVPLMLTIRITLALSYVRLTSSLDGRPLAAAGIVVTGDKAVNIYTSSQTGSIIVKLLPNMPKDKEACAKAPLEAYNRTLTTLLTPLGDSIRRIQESVTTSGGRRQRRSIGAIIGSVALGVATAAQITAASALIQANQNAANILRLKESIAATNEAVHEVTGGLSQLAVAVGKMQQFVNDQLNNTAQELDCIKITQQVGVELNLYLTELTTVFGPQITSPALTQLTVQALYNLAGGNVDYLLTKLGVGNNQLSSLIGSGLITGNPIFYDSQTQLLGIQVTLPSVGNLNNMRATYLETLSVSTTKGFASALVPKVVTQVGSVIEELDTSYCIEADLDLYCTRIVTFPMSPGIYSCLSGNTSACMYSRTEGALTTPYMTLKGSVVANCQMTTCRCADPPGIISQNYGEAVSLIDKHSCNVVSLDGITLRLSGEFDATYQKNISILDSQVLVTGNLDISTELGNVNHSISNALDKLEESNSKLDKVNVKLTSTSALITYIVLTVISLVLGMLSLVLACYLMYKQKAQRKTLLWLGNNTLDQMRATTKM

SEQ ID NO: 27 - protein, 2204 aa, Newcastle disease virus
  L protein
MAGSGPERAEHQIILPESHLSSPLVKHKLLYYWKLTGLPLPDECDFDHLIISRQWKKVLESATPDIERMIKLGRAVHQTLNHNSRITGVLHPRCLEELASIEVPDSTNKFRKIEKKIQIHNTRYGELFTRLCTHVEKKLLGSSWSNNVPRSEEFNSIRTDPAFWFHSKWSTAKFAWLHIKQVQRHLIVAARTRSAVNKLVTLTHKVGQIFVTPELVIVTHTDENKFTCLTQELVLMYADMMEGRDMVSIISSTAAHLRSLSEKIDDILRLVDALARDLGNQVYDVVALMEGFAYGAVQLLEPSGTFAGDFFAFNLQELKDTLTGLLPKDIAESVTHAIATIFSGLEQNQAAEMLCLLRLWGHPLLESRIAAKAVRSQMCAPKMVDFDMILQVLSFFKGTIINGYRKKNAGVWPRVKIDTIYGKVIGQLHADSAEISHDVMLREYKSLSALEFEPCIEYDPVTNLSMFLKDKAIAHPKDNWLASFRRNLLSEDQKKHVREATSTNRLLIEFLESNDFDPYKEMEYLTTLEYLRDDNVAVSYSLKEKEVKVNGRIFAKLTNRLRNCQVMAEGILADQIAPFFQGNGVIQDSISLTKSMLAMSQLSFNSNKKRITDCKERVSSNRNHDPKSKNRRRVATFVTTDLQKYCLNWRYQTVKLFAHAINQLMGLPHFFEWIHLRLMDTTMFVGDPFNPPSDPTDCDLSRVPNDDIYIVSARGGIEGLCQKLWTMISIAAIQLAAARSHCRVACMVQGDNQVIAITREVRSDDSPEMVLTQLHQASDNFFKELIHVNHLIGHNLKDRETIRSDTFFIYSKRIFKDGAILSQVLKNSSKLVLISGDLSENTVMSCANIASTVARLCENGLPKDFCYYLNYLMSCVQTYFDSEFSITNNSQLDSNQSWIEDISFVHSYVLTPAQLGGLSNLQYSRLYTRNIGDPGTTAFAEIKRLEAVGLLSPSIMTNILTRPPGNGDWASLCNDPYSFNFETVASPNIVLKKHTQRVLFETCSNPLLSGVHTEDNEAEEKALAEFLLNQEMIHPRVAHAIMEASSVGRRKQIQGLVDTTNTVIKIALTRRPLGIKRLMRIVNYSSMHAMLFRDDVFSSNRSNHPLVSSDMCSQTLADYARNRSWSPLTGGRKILGVSNPDTIELVEGEILSVSGGCTRCDSGDEQFTWFHLPSNIELNDDTSKNPPMRVPYLGSKTQERRAASLAKIAHMSPHVKAALRASSVLIWAYGDNEINWTAALKIARSRCNISSEYLRLLSPLPTAGNLQHRLDDGITQMTFTPASLYRVSPYIHISNDSQRLFTEEGVKEGNVVYQQIMLLGLSLIESLFPMTTTKTYDEITLHLHSKFSCCIREAPVAVPFELLGVAPELRAVTSNKFMYDPSPVSEGDFARLDLAIFKSYELNLESYSTIELMNVLSISSGKLIGQSVVSYDEDTSIKNDAIIVYDNTRNWISEAQNSDVVRLFEYAALEVLLDCSYQLYYLRVRGLNNIVLYMSDLYKNMPGILLSNIAATISHPVIHSRLNAVGLVNHDGSHQLADTDFIEMSAKLLVSCTRRVVSGLHAGNKYDLLFPSVLDDNLSEKMLQLISQLCCLYTVLFATTREIPKIRGLSAEEKCSVLTEYLLSDAVKPLLGSEQVSSIMSPNIVTFPANLYYMSRKSLNLIREREDRDTILALLFPQEPLLEFPLVRDIGARVKDPFTLQPAAFLQELDLSAPARYDAFTISQAHSEHILPNPEEDHLVRYLFRGIGTASSSWYKASHLLSVPEVRCARHGNSLYLAEGSGAIMSLLELHIPHETIYYNTLFSNEMNPPQRHFGPTPTQFLNSVVFRNLQAEVPCKDGFVQEFRPLWRENTEESDLTSDKAVGYITSVVPYRSVSLLHCDIEIPPGSNQSLLDQLATNLSLIAMHSVKEGGVVIIKVLYAMGYYFHLLMNLFTPCSTKGYILSNGYACRGDMECYLIFVMGYLGGPTFVHEVVRMAKTLVQRHGTLLSKSDEITLTRLFTSQQQRVTDILSSPLPRLMKYLRENIDTALIEAGGQPVRPFCAESLVSTLTDMTQTTQIIASHIDTVIRSVIYMEAEGDLADTVFLFTPYNLSMDGKKRTSLKQCTRQILEVTILGLRVKNLNKVGDVISLVLRGMISLEDLLPLRTYLKCSTCPKYLKAVLGITKLKEMFTDASLLYLTRAQQKFYMKTIGNAVKGYYSNCDS

SEQ ID NO: 28 - DNA, 36 bp, Artificial Sequence
  Primer
acgactcactataggaccaaacagagaatccgtgag

SEQ ID NO: 29 - DNA, 35 bp, Artificial Sequence
  Primer
ccgggaagatccagggcactcttcttgcatgttac

SEQ ID NO: 30 - DNA, 35 bp, Artificial Sequence
  Primer
gggcctgcctcactatggtggtaacatgcaagaag

SEQ ID NO: 31 - DNA, 35 bp, Artificial Sequence
  Primer
tgcatgttaccaccaatgtgtcattgtatcgcttg

SEQ ID NO: 32 - DNA, 37 bp, Artificial Sequence
  Primer
caagaagggagatacgtaatatacaagcgatacaatg

SEQ ID NO: 33 - DNA, 35 bp, Artificial Sequence
  Primer
tcgcttgtatattacttgttgtagcaaagagcacc

SEQ ID NO: 34 - DNA, 38 bp, Artificial Sequence
  Primer
ggcctggatcttcccattatgctgtctgtatacggtgc

SEQ ID NO: 35 - DNA, 38 bp, Artificial Sequence
  Primer
atgccatgccgacccaccaaacaaagacttggtgaatg

SEQ ID NO: 36 - DNA, 24 bp, Artificial Sequence
  Primer
cctatagtgagtcgtattaatttc

SEQ ID NO: 37 - DNA, 20 bp, Artificial Sequence
  Primer
cctggatcttcccgggtcgg

SEQ ID NO: 38 - DNA, 22 bp, Artificial Sequence
  Primer
gggtcggcatggcatctccacc

SEQ ID NO: 39 - DNA, 22 bp, Artificial Sequence
  Primer
gggaagatccaggcctatagtg

SEQ ID NO: 40 - DNA, 35 bp, Artificial Sequence
  Primer
ctctagagtcgacccttctgccaacatgtcttccg

SEQ ID NO: 41 - DNA, 35 bp, Artificial Sequence
  Primer
gggaagcggccgcccgtcggtcagtatccccagtc

SEQ ID NO: 42 - DNA, 37 bp, Artificial Sequence
  Primer
ctctagagtcgaccccagagtgaagatggccaccttc

SEQ ID NO: 43 - DNA, 35 bp, Artificial Sequence
  Primer
gggaagcggccgcccgtagtagtgatcagccattc

SEQ ID NO: 44 - DNA, 35 bp, Artificial Sequence
  Primer
ctctagagtcgacccgggtaggacatggcgggctc

SEQ ID NO: 45 - DNA, 37 bp, Artificial Sequence
  Primer
gggaagcggccgccctgcctttaagagtcacagttac